(19) United States
(12) Patent Application Publication
(10) Pub. No.: US 2007/0088043 A1
(43) Pub. Date: Apr. 19, 2007

(54) Novel HDAC inhibitors

(75) Inventors: Akella Satya Surya Visweswara Srinivas, Tamil Nadu (IN); Kilambi Narasimhan, Tamil Nadu (IN); Lakshmanan Manikandan, Tamil Nadu (IN); Sriram Rajagopal, Tamil Nadu (IN); Thangapazham Selvakumar, Tamil Nadu (IN); Gaddam Om Reddy, Tamil Nadu (IN)

Correspondence Address: OLIFF & BERRIDGE, PLC, P.O. BOX 19928, ALEXANDRIA, VA, 22320, US

(73) Assignee: ORCHID RESEARCH LABORATORIES LIMITED., CHENNAI (IN)

(21) Appl. No.: 11/581,570

(22) Filed: Oct. 17, 2006

(30) Foreign Application Priority Data
Oct. 18, 2005 (IN) 1492/CHE/2005

(51) Int. Cl. A61K 31/496; C07D 417/02

(52) U.S. Cl. 514/254.02; 514/254.04; 544/368; 544/367

(57) ABSTRACT

The present invention relates to novel compounds of the general formula (I), their derivatives, analogs, tautomeric forms, stereoisomers, polymorphs, hydrates, solvates, pharmaceutically acceptable salts and compositions. The present invention more particularly provides novel HDAC inhibitors of the general formula (1). Also included is a method for treatment of cancer, psoriasis, proliferative conditions and conditions mediated by HDAC, in a mammal comprising administering an effective amount of a compound of formula (I) as described above.

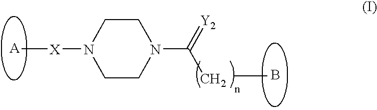

### FIELD OF THE INVENTION

The present invention relates to novel compounds of the general formula (I), their derivatives, analogs, tautomeric forms, stereoisomers, polymorphs, hydrates, solvates, pharmaceutically acceptable salts and compositions. The present invention more particularly provides novel HDAC inhibitors of the general formula (I). (I).

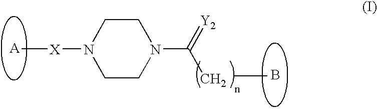

The present invention also provides a process for the preparation of the above said novel compounds of the formula (I), their derivatives, analogs, stereoisomers, polymorphs, hydrates, solvates, pharmaceutically acceptable salts and compositions.

The novel compounds (I) of the present invention are useful for the treatment cancer, which is one of the leading causes of death in the present society. A great deal of effort has been underway to treat various forms of cancer for decades and until recently. Chemoprevention of cancer is receiving its due share of attention.

The first isolation of histone deacetylase was described in 1964 from crude nuclear extracts of cells, but the molecular characterization of isoforms of the enzyme has been achieved recently. Inhibitors of histone deacetylase (HDAC's) are zinc hydrolase's responsible for the deacetylation of N-acetyl lysine residues of histone and nonhistone protein substrates. Human HDAC's are classified into two distinct classes, the HDAC's and sirtuins. The HDAC's are devised into two subclasses based on their similarity to yeast histone deacetylases, RPD 3 (class I includes HDAC 1, 2, 3, 8, and 11) and Hda 1 (class II includes HDAC 4, 6, 7, 9, and 10). All of the HDAC's have a highly conserved zinc dependent catalytic domain. There is growing evidence that the acetylation state of proteins and thus the HDAC enzyme family plays a crucial role in the modulation of a number of biological processes, including transcription and cell cycle.

Transcriptional regulation is a major event in cell differentiation, proliferation and apoptosis. Transcriptional activation of a set of genes determines cell destination and for this reason transcription is tightly regulated by a variety of factors. One of its regulatory mechanisms involved in the process is an alteration in the tertiary structure of DNA, which affects transcription factors to their target DNA regiments. Nucleosomal integrity is regulated by the acetylating status of the core histone, with the result being permissiveness to transcription. The acetylating status of the histone is governed by the balance of activities of the histone acetyl transferase (HAT) and histone deacetylase (HDAC). Recently HDAC inhibitors have been found to arrest growth and apoptosis in several types of cancer cells, including colon cancer, t-cell lymphoma and erythroleukemic cells.

Given that apoptosis is a crucial factor for cancer progression, HDAC inhibitors are promising reagents for cancer therapy as effective inducers of apoptosis.

Several structural classes of HDAC inhibitors have been identified and are reviewed in Marks, P. A. et al., J. Natl. Cancer Inst., 92, (2000), 1210-1215. More specifically WO 98/55449 and U.S. Pat. No. 5,369,108 report alkanoyl hydroxamates with HDAC inhibitory activity.

### BACKGROUND OF THE INVENTION

Cancer is not just one disease, but also a large group of almost one hundred diseases. It's a multifactorial disease. Its two main characteristics are uncontrolled growth of the cells in the body and the ability of these cells to migrate from the original site and spread to distant sites. If the speed is not controlled, cancer can result to death. Cancer is the growth of tumor cells that interfere with the growth of healthy cells. Cancer, by definition is a disease of the genes. A gene is a small part of DNA, which is the master molecule of the cell. Genes make “Proteins”, which are the ultimate workhorses of the cells, these Proteins allow our bodies to carry out all the many processes that permit us to breath, think, move . . . etc.

Cancer is believed to be the number one cause of premature death in industrialized nations. Cancer incidence is growing throughout the world and is the second leading cause of death among Americans. In the male population throughout the world, rates of occurrence of prostrate cancers are extremely high followed by lung, colon, oral and melanoma. In the female population, incidence of breast cancer is high followed by colon, lung, uterus, cervix, lymphoma, and ovarian cancers.

The cells in the body are growing, dividing and replacing themselves. Many genes produce proteins that are involved in controlling the processes of cell growth and division. An alteration (Mutation) to the DNA molecule can disrupt the genes and produce faulty proteins. This causes the cell to become abnormal and lose its resistance and growth. The abnormal cell begins to divide uncontrollably and eventually forms a new growth known as a tumor or neoplasm. In healthy individual, the immune system can recognize the tumor cells and destroy them before they get a chance to divide.

Tumors are of two types, benign and malignant. A benign tumor is slow growing, does not spread to the surrounding tissues, and once it is removed, it does not usually recover. A malignant tumor, on the other hand, invades surrounding tissues and spreads to other parts of the body. Even after the malignant tumor is removed, it generally recovers. The most common cancers are Skin cancer, Lung cancer, Colon cancer, Breast cancer (in women) and Prostate cancer (in men). In addition, cancers of the Kidneys, Ovaries, Uterus, Pancreas, Bladder, Rectum and Blood and Lymph node cancer (Leukemia and Lymphoma) are also included among the 12 major cancers that affect most Americans. Tobacco, alcohol, diets, sexual and reproductive behavior, infectious agents, Family history, environment and pollution are the main sources for getting cancer. The usual treatments of cancer are surgery, chemotherapy (treatment with anticancer drugs) and radiation or some combination of these methods. Prevention of cancer by eating plenty of vegetables and fruits, exercising vigorously for at least 20 minutes every day, avoiding excessive weight gain, avoiding tobacco (even second hand smoke), decreasing or avoiding consumption of animal fats and red meats, avoiding excessive amounts of alcohol, avoiding the midday sun, when the sun rays are the strongest, avoiding risky sexual practices and avoiding known carcinogens in the environment or work place.

Histone hyperacetylation by HDAC inhibition neutralizes the positive charge of the lysine side chain, and is thought to be associated with change of the chromatin structure and the consequential transcriptional activation of a number of genes. One important outcome of histone hyperacetylation is induction of the Cyclin-dependent kinase inhibitory protein, P21, which causes cell cycle arrest. Indeed, HDAC inhibitors such as Trichostatin A (TSA) and Suberoylanilide. Hydroxamic acid (SAHA) has been reported to inhibit cell growth, induce terminal differentiation in tumor cells and prevent the formation of tumors in mice. Therefore, HDAC's have been viewed as attractive targets for anticancer drug development.

The present invention relates to potentially pharmaceutical compositions and in particular to new molecules as active ingredients, that are used in particular as anticancer agents. Compounds of the general formula (1) or pharmaceutically acceptable salts thereof according to the present invention have an ability of inhibiting histone deacetylating enzyme and of inducing differentiation and are useful as therapeutic or ameliorating agent for diseases that are involved in cellular growth such as malignant tumors, autoimmune diseases, skin diseases, infections etc.

Few prior art references, which disclose the closest compounds, are given here:

I. US 2005/0143385 discloses compounds of formula 1. 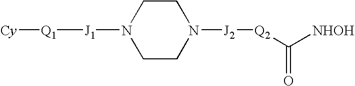
wherein: Cy is Cyclyl group (C₃₋₂₀ carbocyclyl, C₃₋₂₀ heterocyclyl or C₅₋₂₀ aryl and is optionally substituted), Q₁ represents covalent bond or cyclyl leader group (C₁₋₇ alkylene, C₁₋₇ alkylene-X— C₁₋₇ alkylene, —X— C₁₋₇ alkylene, or C₁₋₇ alkylene-X—), wherein X is —O— or —S— and is optionally substituted; J₁ represents covalent bond or —C(═O)—; J₂ is independently —C(═O)— or —S(═O)₂—; Q₂ is independently optionally substituted C₄₋₈ alkylene, C₅₋₂₀ arylene, C₅₋₂₀ arylene-C₁₋₇ alkylene, C₁₋₇ alkylene-C₅₋₂₀ arylene, or C₁₋₇ alkylene-C₅₋₂₀ arylene-C₁₋₇ alkylene.

The present invention pertains to pharmaceutical compositions containing such compounds and their use, both in vitro and in vivo, to inhibit HDAC, and in the treatment of conditions mediated by HDAC, cancer, proliferative conditions, psoriasis, etc.
II. U.S. Pat. No. 6,638,530 D1 discloses benzamide derivatives of the formula (1) 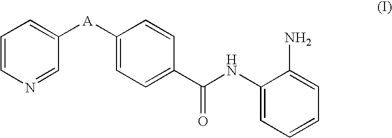
wherein A represents a structure represented by any one of the following

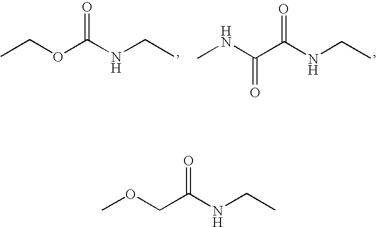

The objective of the present invention is to provide formulations with increased solubility and improved oral absorptivity of benzamide derivatives of the formula (1) and their pharmaceutically acceptable salts that are useful as histone deacetylase inhibitors, and to provide injections containing the active ingredient at high concentrations.
III. WO 2005/065779 A1 discloses compound of the formula 1. 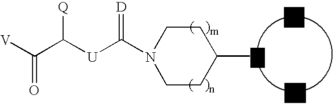
wherein: V represents —N(R¹)(R²) or OR⁴; wherein R¹ and R² are each independently L¹, L¹ is selected from the group consisting of H, C1-6 alkyl, phenyl, pyridyl, pyrimidinyl, morpholinyl and the like. Q is Q′ or Q″; wherein Q, is (S^{y})ₛR³; and Q″ is NH(S^{y})SR³ wherein S^{y} is C₁₋₃alkylene or C₁₋₃alkylidine and s is 0 or 1 and R³ is R^{3a} or R^{3b} represents heterocycle containing one to three of the same or different heteroatoms selected from the group consisting of O, N and S. U is CH₂. D is O. A is N. m and n are independently 0,1 or 2; G, J and E together form A^{x} or A^{y}. p is 0 or 1. A^{y} is a 4 to 6 membered heterocycle containing one to three heteroatoms selected from the group consisting of O, N and S;

The present invention relates to compounds of Formula (1) as antagonists of calcitonin gene-related peptide receptors pharmaceutical compositions comprising, methods for treatment using them and their use in therapy for treatment of neurogenic vasolidation, neurogenic inflammation, migraine and other headaches, thermal injury, circulatory shock, flushing associated with menopause, airway inflammatory diseases, such as asthma and chronic obstructive pulmonary disease (COPD), and other conditions the treatment of which can be effected by the antagonism of CGRP-receptors.

### OBJECTIVE OF THE INVENTION

Due to unmet medical needs and also as all of us know, cancer is one of the leading causes of death in the present society, we focused our attention to identify novel small molecule anticancer agents, particularly focusing on HDAC inhibitors. Our sustained efforts have resulted in novel anticancer agents of the formula (I). Histone acetylation and deacetylation play an essential role in modifying chromatin structure and regulating gene expression in eukaryotic cells. Hyper acetylated histones are generally found in transcriptionally active genes and in transcriptionally silent regions of the genome. Key enzymes, which modify histone proteins and thereby regulate gene expression, are histone acetyl transferases (HATs) and histone deacetylases (HDACs). Compounds able to inhibit HDAC activity i.e. HDAC inhibitors such as Trichostatin A (TSA), Trapoxin (TPX), Suberoylanilide hydroxamic acid (SAHA), Sodium butyrate (NaB), Sodium valproate (VPA), Cyclic hydroxamic acid containing peptides (CHAPs), Depsipeptide FK-228 and MS-275 can de-repress these genes, resulting in antiproliferative effects in vitro and anti tumor effects in vivo.

### SUMMARY OF THE INVENTION

The present invention relates to novel substituted HDAC inhibitors of the general formula (I), 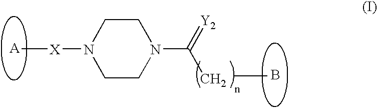
their derivatives, analogs, tautomeric forms, stereoisomers, polymorphs, solvates, pharmaceutically acceptable salts and compositions, wherein A represents substituted or unsubstituted groups selected from aryl, aralkyl, heterocyclyl, heteroaryl and benzo fused heteroaryl; wherein X represents the groups represented by 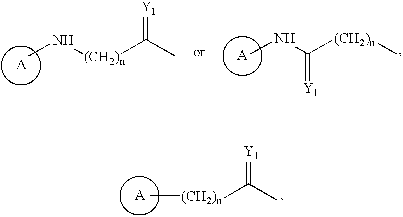 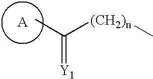
or X and A are fused to form a cyclic structure; wherein Y₁ and Y₂ may be same or different and independently represent oxygen or sulphur; wherein B represents one or more substituted alkyl group, hydroxamic acid group or the thioate, thiol, substituted or unsubstituted hydroxyl amine group (O-acetyl, N-acetyl, O-benzyl both or either of any two combination), substituted hydroxyl, hydroxamic acid group (O-acetyl, N-acetyl, O-benzyl both or either of any two combination), carbonyl or amino group substituted with acetyl group or amino acids or their corresponding hydroxamic acids or their esters (viz. isomers of alanine, cystine, glycine, valine, methionine, serine, tyrosine, isonipecolic acid, proline, and the like.). n is an integer in the range of 0 to 7.

### DETAILED DESCRIPTION OF THE INVENTION

Suitable groups represented by A represents substituted or unsubstituted groups selected from aryl group such as phenyl, naphthyl and the like; arylalkyl such as benzyl, phenylethyl, phenylpropyl and the like; heterocyclyl groups such as pyrrolidinyl, thiazolidinyl, oxazolidinyl, morpholinyl, thiomorpholinyl, piperidinyl, piperazinyl; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like; benzofused heteroaryl groups such as indolyl, indolinyl, benzothiazolyl, quinoline, quinoxaline, quinazoline, acridine, phenazine pteridinyl, phenoxazine, phenothiazine, carbazolyl and the like.

The group X is represented by 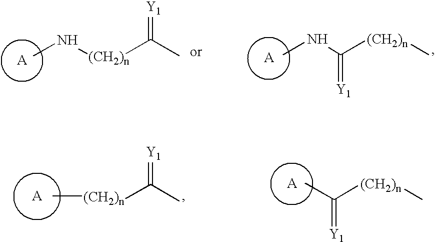
or X and A are fused to form a cyclic structure;

Y₁ and Y₂ may be same or different and independently represent oxygen or sulphur.

Wherein B represents one or more substituted, linear or branched alkyl groups substituted by groups such as amino, hydroxyl, thiol and the like; thioate, thiol, substituted or unsubstituted hydroxyl amine group (O-acetyl, N-acetyl, O-benzyl both or either of any two combination), substituted or unsubstituted hydroxamic acid group (O-acetyl, N-acetyl, O-benzyl both or either of any two combination), carbonyl or amino group substituted with acetyl group or amino acids or their corresponding hydroxamic acids or their esters (viz. isomers of alanine, cystine, glycine, valine, methionine, serine, tyrosine, isonipecolic acid, proline, and the like.) and n is an integer in the range of 0 to 7.

Suitable groups substituted on A with one or more same or different substituents selected from a group consisting of halogens (fluorine, chlorine, bromine, iodine), hydroxy, nitro, cyano, azido, nitroso, amino, hydrazine, formyl, alkyl, haloalkyl, haloalkoxy, cycloalkyl, aryl (may be further substituted), alkoxy, aryloxy, acyl, acyloxy, acyloxyacyl, heterocyclyl, heteroaryl (may be further substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives.

Furthermore A which is a cyclic ring represents substituted or unsubstituted 5 to 10 membered ring systems, and also the rings may be monocyclic or bicyclic, saturated or partially saturated or aromatic containing 1 to 4 hetero atoms selected from O, S and N and the like.

Definitions:

As used throughout the specification and the appended claims the following terms have the following meanings:

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl and tert-butyl,

The term “alkoxy” as used herein, means an alkyl group, as defined herein, appended to the parent molecular moiety through an oxygen atom. Representative examples of alkoxy include, but are not limited to, methoxy, ethoxy, n-propoxy, iso-propoxy and tert-butyl.

The term analog includes a compound, which differs from the parent structure by one or more C, N, O or S atoms. Hence, a compound in which one of the N atoms in the parent structure is replaced by an S atom is an analog of the former.

The term stereoisomer includes isomers that differ from one another in the way the atoms are arranged in space, but whose chemical formulas and structures are otherwise identical. Stereoisomers include enantiomers and diastereoisomers.

The term tautomers include readily interconvertible isomeric forms of a compound in equilibrium. The enol-keto tautomerism is an example.

The term polymorphs include crystallographically distinct forms of compounds with chemically identical structures.

The term pharmaceutically acceptable solvates includes combinations of solvent molecules with molecules or ions of the solute compound.

The term derivative refers to a compound obtained from a compound according to formula (I), an analog, tautomeric form, stereoisomer, polymorph, hydrate, pharmaceutically acceptable salt or pharmaceutically acceptable solvate thereof, by a simple chemical process converting one or more functional groups, such as, by oxidation, hydrogenation, alkylation, esterification, halogenation, and the like.

Pharmaceutically acceptable salts forming part of this invention include base addition salts such as alkali metal salts like Li, Na, and K salts; alkaline earth metal salts like Ca and Mg, salts of organic bases such as lysine, arginine, guanidine, diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

### Particularly Useful Compounds According to the Invention Include

1. N-{2-[4-(5-thioacetylpentanoyl)piperazin-1-yl]-2-oxoethyl}4-methyl-1,3-benzo thiazol-2-amine;
2. N-{2-[4-(6-thioacetylhexanoyl)piperazin-1-yl]-2-oxoethyl}-morpholine (2);
3. N-{2-[4-(6-thioacetylhexanoyl)piperazin-1-yl]-2-oxoethyl}-6-methyl-1,3-benzo thiazol-2-amine;
4. 2-[4-(5-Thioacetylpentanoyl)piperazin-1-yl]-N-(4-methyl-1,3-thiazol-2-yl)acetamide;
5. 2-[4-(6-Thioacetylhexanoyl)piperazin-1-yl]-N-(4-nitro-1,3-thiazol-2-yl)acetamide;
6. 2-[4-(6-Thioacetylhexanoyl)piperazin-1-yl]-N-(1,3-benzothiazol-2-yl)acetamide;
7. 2-[4-(5-Thioacetylpentanoyl)piperazin-1-yl]-N-(1,3-thiozole-2-yl)acetamide;
8. 2-[4-(5-Thioacetylpentanoyl)piperazin-1-yl]-N-(1,3-benzothiazol-2-yl)acetamide;
9. 2-[4-(5-Thioacetylpentanoyl)piperazin-1-yl]-N-(4-nitro-1,3-thiazol-2-acetamide;
10. 2-[4-(6-Thioacetylhexanoyl)piperazin-1-yl]-N-(4-methyl-1,3-benzothiazol-2-yl)acetamide;
11. 2-[4-(5-Thioacetylpentanoyl)piperazin-1-yl]-N-(6-methyl-1,3-benzothiazol-2-yl)acetamide;
12. 2-[4-(6-Thioacetylhexanoyl)piperazin-1-yl]-N-(6-methyl-1,3-benzothiazol-2-yl)acetamide;
13. 2-[4-(5-Thioacetylpentanoyl)piperazin-1-yl]-N-(4-methyl-1,3-benzothiazol-2-yl )acetamide;
14. 2-[4-(5-Thioacetylpentanoyl)piperazin-1-yl]-N-(4-tertiarybutyl-1,3-thiazol-2-yl)acetamide;
15. 2-[4-(5-Thioacetylpentanoyl)piperazin-1-yl]-N-(6-ethoxy-1,3-benzothiazol-2-yl)acetamide;
16. 2-[4-(5-Thioacetylpentanoyl)piperazin-1-yl]-N-(phenyl-2-yl)acetamide;
17. 2-[4-(6-Thioacetylhexanoyl)piperazin-1-yl]-N-(4-methyl-1,3-thiazol-2-yl)acetamide;
18. 2-[4-(6-Thioacetylhexanoyl) piperazin-1-yl]-N-(phenyl-2-yl)acetamide;
19. 2-[4-(6-Thioacetylhexanoyl)piperazin-1-yl]-N-(morpholine)acetamide;
20. 2-[4-(5-Thioacetylpentanoyl)piperazin-1-yl]-N-(3,5-dimethoxybenzyl)-2-yl)acetamide;
21. 2-[4-(6-Thioacetylhexanoyl)piperazin-1-yl]-N-(4-methoxy-1,3-benzothiazol-2-yl)acetamide;
22. 2-[4-(6-Thioacetylhexanoyl)piperazin-1-yl]-N-(6-fluorophenyl-2-yl)acetamide;
23. 2-[4-(5-Thioacetylpentanoyl)piperazin-1-yl]-N-(6-fluorophenyl-2-yl)acetamide;
24. N-1,3-benzothiazol-2-yl-2-[4-(6-mercaptohexanoyl)piperazin-1-yl]acetamide;
25. N-6-methyl-1,3-benzothiazol-2-yl-2-[4-(6-mercaptohexanoyl)piperazin-1-yl]acetamide;
26. N-1,3-benzothiazol-2-yl-2-{6-[6 (hydroxyamino)hexanoyl]piperazin-1-yl}acetamide;
27. N-4-methyl-1,3-benzothiazol-2-yl-2-{6-[6 (hydroxyamino)hexanoyl]piperazin-1-yl}acetamide;
28. N-4-methoxy-1,3-benzothiazol-2-yl-2-{6-[6 (N-acetyl-O-benzyl)hexanoyl]piperazin-1-yl}acetamide;
29. 8-{4-[2-(1,3-Benzothiazol-2-ylamino)-2-oxoethyl]piperazin-1-yl}-N-hydroxy-8-oxo octanamide;
30. 8-{4-[2-(6-Methyl-1,3-benzothiazol-2-ylamino)-2-oxoethyl]piperazin-1-yl}-N—N-[(2-amino-3-hydroxypropanoyl)amino]-8-oxo octanamide;
31. 8-{4-[2-(Phenyl-2-ylamino)-2-oxoethyl]piperazin-1-yl}-N-hydroxy-8-oxo octanamide;
32. 8-{4-[2-(6-Ethoxy-1,3-benzothiazol-2-ylamino)-2-oxoethyl]piperazin-1-yl}-N-hydroxy-8-oxo octanamide;
33. 8-{4-[2-(4-Methyl-1,3-thiazol-2-ylamino)-2-oxoethyl]piperazin-1-yl}-N-hydroxy-8-oxo octanamide;
34. 8-{4-[2-(1,3-Thiazol-2-ylamino)-2-oxoethyl]piperazin-1-yl}-N-hydroxy-8-oxo octanamide;
35. 8-{4-[2-(6-Methyl-1,3-benzothiazol-2-ylamino)-2-oxoethyl]piperazin-1-yl}-N-hydroxy-8-oxo octanamide;
36. 8-{4-[2-(3-Fluorophenyl-2-ylamino)-2-oxoethyl]piperazin-1-yl}-N-hydroxy-8-oxo octanamide;
37. 8-{4-[2-(6-Ethoxy-1,3-benzothiazol-2-ylamino)-2-oxoethyl]piperazin-1-yl}-N-acetyl-N-hydroxy-8-oxo octanamide;
38. 8-{4-[2-(6-Methyl-1,3-benzothiazol-2-ylamino)-2-oxoethyl]piperazin-1-yl}-N-acetyl-N-hydroxyacetyl-8-oxo octanamide;
39. 8-{4-[2-(1,3-Benzothiazol-2-ylamino)-2-oxoethyl]piperazin-1-yl}-N-acetyl-N-hydroxy-8-oxo octanamide;
40. 8-{4-[2-(4-Methyl-1,3-benzothiazol-2-ylamino)-2-oxoethyl]piperazin-1-yl}-N-hydroxybenzyl-8-oxo octanamide;
41. 8-{4-[2-(4-Methyl-1,3-benzothiazol-2-ylamino)-2-oxoethyl]piperazin-1-yl}-N-acetyl-N-hydroxybenzyl-8-oxo octanamide;
42. 6-{4-[2-(1,3-Benzothiozol-2-ylamino)-2-oxoethyl]piperazin-1-yl}-N-{[2-amino-4-(methylthio)butanoyl]amino}hexanamide;
43. 6-{4-[2-(1,3-Benzothiozol-2-ylamino)-2-oxoethyl]piperazin-1-yl}-N-(acetylamino)hexanamide;
44. 6-{4-[2-(1,3-Benzothiozol-2-ylamino)-2-oxoethyl]piperazin-1-yl}-N-[(2-amino-3-hydroxypropanoyl)amino]hexanamide;
45. 2-[4-(2-Amino-3-hydroxypropanoyl)piperazin-1-yl]-N-1,3-benzothiazol-2-ylacetamide;
46. 6-{4-[2-(1,3-Benzothiozol-2-ylamino)-2-oxoethyl]piperazin-1-yl}-N-[(2-amino-3-methylbutyl)amino]hexanamide;
47. 6-{4-[2-(1,3-Benzothiozol-2-ylamino)-2-oxoethyl]piperazin-1-yl}-N-(aminoacetyl)hexanamide and
48. 2-[4-(2-Amino-3-hydroxypropanoyl)piperazin-1-yl]-N-1,3-thiazol-2-ylacetamide.

According to another feature of the present invention, there is provided a process as shown in the following schemes, for the preparation of compounds of the formula (I), wherein all the groups are as defined earlier.

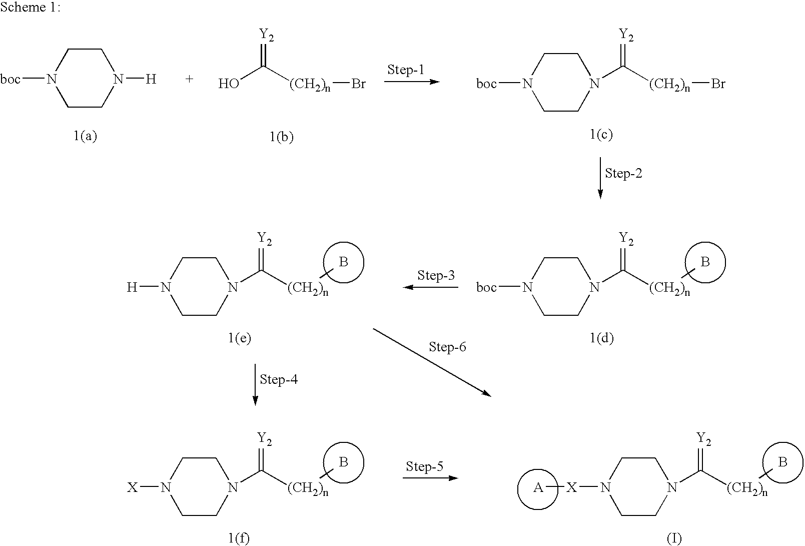

Condensation of the compound of formula 1(a) and the compound of formula 1(b) using EDCI and HOBt, yielded a compound of formula 1(c), wherein Y₂ is as defined earlier.

Further the compound of formula 1(c) is converted to the compound of formula 1(d) with either potassium thioacetate or hydroxamic acid derivatives, wherein B is as defined earlier.

The compound of formula 1(d) was further converted to the compound of formula 1(e) by reaction with acids like trifluoroacetic acid or hydrochloric acid and the like.

The compound of formula 1(e) was reacted with aromatic or non-aromatic or alkylic or hetero aromatic amino acetyl bromide compounds give the compound of formula 1, all the terms are individually described as earlier.

The compound of formula 1(e) was reacted with bromo acetyl bromide or chloro acetyl chloride to give the compound of formula 1(f), which was further reacted with different amines to give the compounds of formula I all the terms are individually described as earlier.

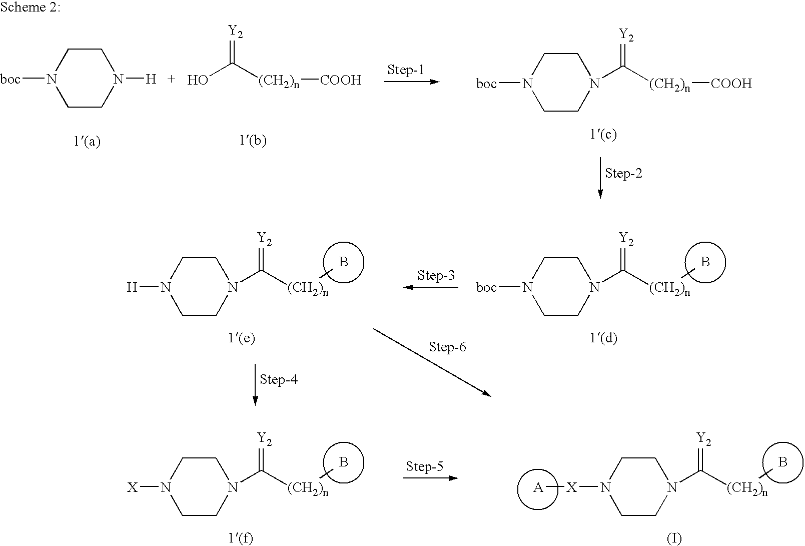

Condensation of the compound of formula 1′(a) and the compound of formula 1′(b) using BOP and HOBt, yielded a compound of formula 1′(c), wherein Y₂ is as defined earlier.

Further the compound of formula 1′(c) is converted to the compound of formula 1′(d) with hydroxamic acid derivatives, where in B is as defined earlier. The compound of formula 1′(d) was further converted to the compound of formula 1′(e) by reaction with acids like trifluoroacetic acid or hydrochloric acid and the like.

The compound of formula 1′(e) was reacted with aromatic or non-aromatic or alkylic or hetero aromatic amino acetyl bromide compounds give the compound of formula I, all the terms are individually described as earlier.

The compound of formula 1′(e) was reacted with bromo acetyl bromide or chloro acetyl chloride to give the compound of formula 1′(f), which was further reacted with different amines to give the compounds of formula 1, all the terms are individually described as earlier.

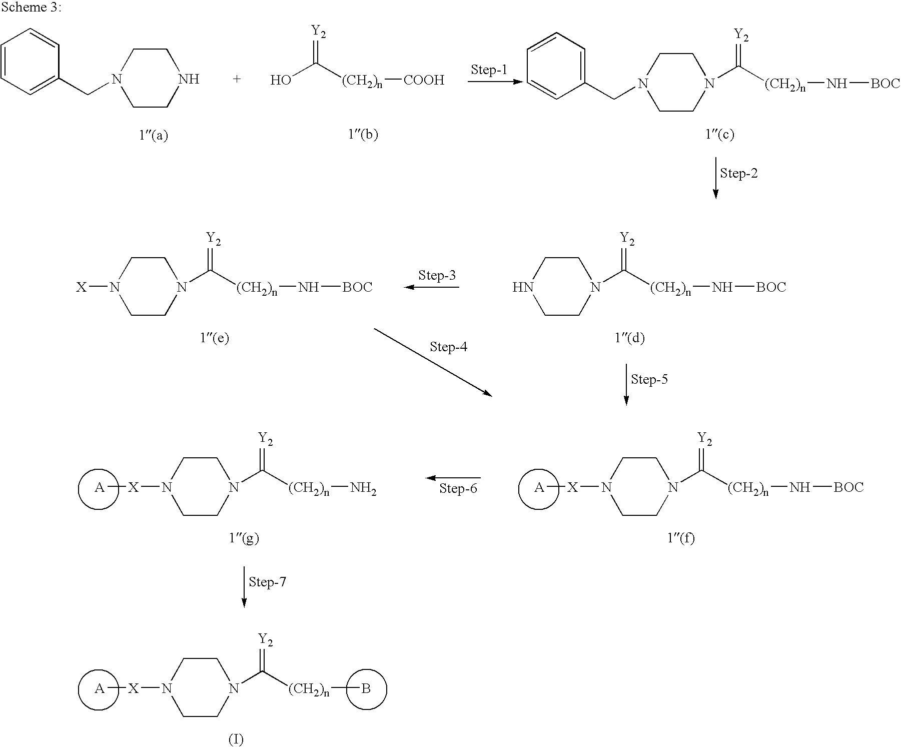

Condensation of the compound of formula 1″(a) and the compound of formula 1″(b) using BOP and HOBt, yielded a compound of formula 1″(c), wherein Y₂ is as defined earlier.

Further the compound of formula 1″(c) is converted to the compound of formula 1″(d) by reaction with reagents like palladium carbon and hydrogen and the like.

The compound of formula 1″(d) was reacted with bromo acetyl bromide or chloro acetyl chloride to give the compound of formula 1″(e), which was further reacted with different amines to give the compounds of formula 1″(f).

The compound of formula 1″(d) was reacted with aromatic or non-aromatic or alkylic or hetero aromatic amino acetyl bromide compounds give the compound of formula 1″(f).

Further the compound of formula 1″(f) is converted to the compound of formula 1″(g) by reaction with acids like trifluoroacetic acid or hydrochloric acid and the like.

The compound of formula 1″(g) is reacted with various amino acids or their derivatives or aromatic or non-aromatic or alkylic or hetero aromatic acids give the compound of formula I, all the terms are individually described as earlier.

It is appreciated that in any of the above-mentioned reactions, any reactive group in the substrate molecule may be protected according to conventional chemical practice. Suitable protecting groups in any of the above-mentioned reactions are those used conventionally in the art. The methods of formation and removal of such protecting groups are those conventional methods appropriate to the molecule being protected. Protecting groups are removed under conditions, which will not affect the remaining portion of the molecule.

The pharmaceutically acceptable salts are prepared by reacting the compound of formula (I) with 1 to 10 equivalents of a base such as sodium hydroxide, sodium methoxide, sodium hydride, potassium t-butoxide, calcium hydroxide, magnesium hydroxide and the like, in solvents like ether, tetrahydrofuran, methanol, t-butanol, dioxane, isopropanol, ethanol etc. Mixture of solvents may be used. Organic bases such as diethanolamine, α-phenylethylamine, benzylamine, piperidine, morpholine, pyridine, hydroxyethylpyrrolidine, hydroxyethylpiperidine, choline, guanidine and the like, ammonium or substituted ammonium salts, aluminum salts. Amino acids such as glycine, alanine, cysteine, lysine, arginine, phenylalanine etc may be used for the preparation of amino acid salts. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid, oxalic acid and the like in solvents like ethyl acetate, ether, alcohols, acetone, tetrahydrofuran, dioxane etc. Mixture of solvents may also be used.

It should be noted that compounds of the invention may contain groups that may exist in tautomeric forms, and though one form is named, described, displayed and/or claimed herein, all the tautomeric forms are intended to be inherently included in such name, description, display and/or claim.

The stereoisomers of the compounds forming part of this invention may be prepared by using the reactants in their single enantiomeric form. The stereoisomers of the compounds can also be made by conducting the reaction in the presence of reagents or catalysts in their single enantiomer form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or by using chiral bases such as brucine, cinchona alkaloids, their derivatives and the like. Commonly used methods are compiled by Jaques et al in “Enantiomers, Racemates and Resolution” (Wiley Interscience, 1981).

Prodrugs of the compounds of formula (I) are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art.

Various polymorphs of the compounds of the general formula (I), forming part of this invention may be prepared by crystallization of the compounds of formula (I) under different conditions. For example, using different commonly used solvents, or their mixtures for recrystallization; crystallizations at different temperatures; various modes of cooling, ranging from very fast to very slow cooling during crystallizations. Heating or melting the compounds followed by cooling gradually or immediately, one can also obtain polymorphs. The presence of polymorphs may be determined by solid probe NMR spectroscopy, IR spectroscopy, differential scanning calorimetry and powder X-ray diffraction or other such techniques.

The present invention also provides a pharmaceutical composition, containing one or more of the compounds of the general formula (I) as defined above, their derivatives, analogs, tautomeric forms, stereoisomers, polymorphs, hydrates, metabolites, prodrugs, pharmaceutically acceptable salts, and pharmaceutically acceptable solvates in combination with the usual pharmaceutically employed carriers, diluents and the like, useful for the treatment and/or prevention of cancer, psoriasis, proliferative conditions, conditions mediated by HDAC and diseases involved in cellular growth such as malignant tumors, autoimmune diseases, skin diseases and infections.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art. The amount of the active ingredient in the composition may be less than 70% by weight. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

The pharmaceutical compositions of the present invention are effective in treating and/or preventing cancer, psoriasis, proliferative conditions, conditions mediated by HDAC and diseases involved in cellular growth such as malignant tumors, autoimmune diseases, skin diseases and infections. Generally, the effective dose for treating a particular condition in a patient may be readily determined and adjusted by the physician during treatment to alleviate the symptoms or indications of the condition or disease. Generally, a daily dose of active compound in the range of about 0.01 to 1000 mg/kg of body weight is appropriate for administration to obtain effective results. The daily dose may be administered in a single dose or divided into several doses. In some cases, depending upon the individual response, it may be necessary to deviate upwards or downwards from the initially prescribed daily dose. Typical pharmaceutical preparations normally contain from about 0.2 to about 500 mg of active compound of formula I and/or its pharmaceutically active salts or solvates per dose.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition. Thus compounds of the present invention can be used in the treatment of cancer, psoriasis, as a monotherapy, or also in combination of these HDAC inhibitors with other clinically relevant cytotoxic agents or non-cytotoxic agents.

The term “therapeutically effective amount” or “effective amount” refers to that amount of a compound or mixture of compounds of formula (I) that is sufficient to effect treatment, as defined below, when administered alone or in combination with other therapies to an animal in need of such treatment. More specifically, it is that amount that is sufficient to treat cancer and/or psoriasis.

The term “animal” as used herein is meant to include all mammals, and in particular humans. Such animals are also referred to herein as subjects or patients in need of treatment. The therapeutically effective amount will vary depending upon the subject and disease condition being treated, the weight and age of the subject, the severity of the disease condition, the particular compound of formula (I) chosen, the dosing regimen to be followed, timing of administration, the manner of administration and the like, all of which can readily be determined by one of ordinary skill in the art.

The term “treatment” or “treating” means any treatment of a disease in a mammal, including:

a) Preventing the disease, that is, causing the clinical symptoms of the disease not to develop;

b) Inhibiting the disease, that is, slowing or arresting the development of clinical symptoms; and/or

c) Relieving the disease, that is, causing the regression of clinical symptoms.

From the foregoing description, one skilled in the art can easily ascertain the essential characteristics of this invention, and without departing from the spirit and scope thereof, can make various changes and modifications of the invention to adapt it to various usages and conditions.

The present invention is provided by the examples given below, which are provided by the way of illustration only, and should not be considered to limit the scope of the invention. Variation and changes, which are obvious to one skilled in the art, are intended to be within the scope and nature of the invention, which are defined in the appended claims.

### EXAMPLE 1

### Synthesis of N-{2-[4-(5-thioacetylpentanoyl)piperazin-1-yl]-2-oxoethyl}-1,3-benzothiazol-2-amine

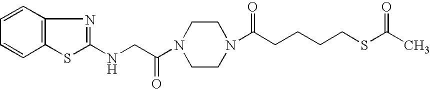 Step-I

### Synthesis of t-Butyl 4-(5-bromopentanoyl)piperazine-1-carboxylate

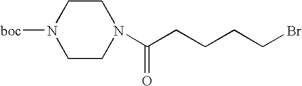

5-bromopentanoic acid (5.83 g, 32.22 mmol), tert-butyl piperazine-1-carboxylate (3 g, 16.11 mmol), 1-(3-Dimethylaminopropyl)-3-ethylcarbodiimide hydrochloride (EDCI) (5.56 g, 29 mmol) and 1-hydroxybenzotriazole (HOBt) (0.87 g, 6.44 mmol) were taken up in THF (150 ml), and stirred for 15 minutes. Triethylamine (6.73 ml, 48.33 mmol) was added drop wise to the above, and stirring was continued for further 6 hours. The reaction mixture was poured into water and extracted thrice with ethyl acetate. The combined organic layer was washed with brine, dried over anhydrous Na₂SO₄ and concentrated under reduced pressure. The resulting crude material was purified by column chromatography using ethyl acetate and n-hexane mixture to give pure tert-butyl 4-(5-bromopentanoyl)piperazine-1-carboxylate (2.6 g, 46.3%).

Step-II

### Synthesis of t-Butyl 4-(5-thioacetyl pentanoyl)piperazine-1-carboxylate

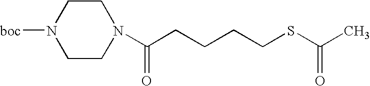

To a solution of t-Butyl 4-(5-bromopentanoyl)piperazine-1-carboxylate (2.5 g, 7.16 mmol) in ethanol (25 ml) at room temperature was added potassium thioacetate (KSAc) (1.23 g, 10.75 mmol) and was stirred for 15 minutes. The reaction mixture was heated to 50° C. and maintained at the same temperature for 7 hours; subsequently the ethanol was completely distilled out, and water was added. It was extracted three times with ethyl acetate; the combined organic layer was washed with brine, dried over anhydrous Na₂SO₄ and concentrated under reduced pressure. The resulting crude material was purified by column chromatography using ethyl acetate and n-hexane mixture to give pure tert-butyl 4-(5-thioacetyl pentanoyl)piperazine-1-carboxylate (2 g, 81.3%).

Step-III

### Synthesis of S-(5-oxo-5-piperazin-1-ylpentyl)ethane thioate

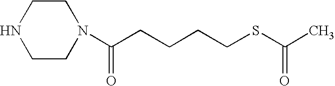

TFA (6 ml, 78.45 mmol) was added to tert-butyl 4-(5-thioacetyl pentanoyl)piperazine-1-carboxylate (1.8 g, 5.23 mmol), and the reaction mixture was stirred for 3 hours. Subsequently the reaction mixture was diluted with ethyl acetate; NaHCO₃ (4.39 g, 52.3 mmol) was added and it was stirred for another 1 hour, and then filtered through a Hirsch funnel. The resulting filtrate was concentrated under reduced pressure, and was used as such for the next reaction.

Step-IV

### Synthesis of S-{5-[4-(bromoacetyl)piperazin-1-yl]-5-oxopentyl}ethane thioate

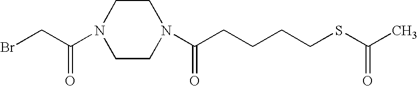 To a solution of S-(5-oxo-5-piperazin-1-ylpentyl)ethane thioate (1 g, 4.1 mmol) in dichloromethane (20 ml) at 0-5° C., was added drop wise, bromoacetyl bromide (0.43 ml, 4.92 mmol) and triethylamine (1.41 ml, 8.2 mmol) in dichloromethane (each 2 ml) simultaneously. The reaction mixture was stirred for 2.5 hours, subsequently the dichloromethane was removed completely and water was added. The reaction mixture was extracted thrice with ethyl acetate; the combined organic layer was washed with brine, dried over anhydrous Na₂SO₄ and concentrated under reduced pressure to afford a concentrate, which was used further for the next reaction.
Step-V

### Synthesis of N-{2-[4-(5-thioacetylpentanoyl)piperazin-1-yl]-2-oxoethyl}-1, 3-benzothiazol-2-amine

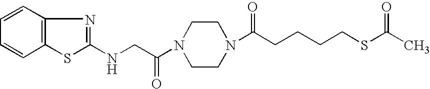

To a solution of S-{5-[4-(bromoacetyl)piperazin-1-yl]-5-oxopentyl}ethane thioate (0.3 g, 0.82 mmol) in acetone (20 ml) was added NaI (0.15 g, 0.985 mmol), and the reaction mixture was stirred for 2 hours. 2-Amino-1,3-benzothiazole (0.094 g, 0.82 mmol) in acetone (20 ml) and triethylamine (0.2 ml) was then added and the stirring was continued for 4 hours. Subsequently the acetone was stripped off, water was added and the reaction mixture was extracted with ethyl acetate. The combined organic layer was washed with brine, dried over anhydrous Na₂SO₄ and concentrated under reduced pressure. The resulting crude material was purified by column chromatography using dichloromethane and methanol mixture to gave pure yellow colour solid N-{2-[4-(5-thioacetylpentanoyl)piperazin-1-yl]-2-oxoethyl}-1,3-benzothiazol-2-amine (0.1 g, 30.49%). ¹H NMR (400 MHz, CDCl₃): δ1.55 (4H, m, —CH₂), 2.33 (3H, s, —CH₃), 2.34 (2H, t, —CH₂), 2.85 (4H, m, —CH₂), 3.45 (4H, m, —CH₂), 3.58 (4H, m, —CH₂), 4.84 (2H, t, —CH₂), 6.96 (2H, m, —CH), 7.15 (1H, d, —CH), 7.39 (1H, d, —CH), 8.25 (1H, brs, —NH); M⁺+1 found at 435.1.

The Following Compounds were Synthesized Using the Procedure Given in the Example 1.

| Ex. | Structure | Analytical Data |
|---|---|---|
| 2 | 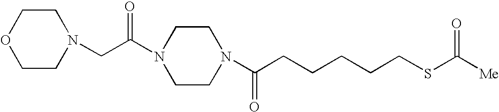 | ¹H NMR (400 MHz, CDCl₃): δ1.57-1.68 (6H, m, —CH₂), 2.32 (3H, s, —CH₃), 2.35 (2H, t, —CH₂), 2.53 (4H, m, —CH₂), 2.87 (2H, t, —CH₂), 3.22 (2H, s, —CH₂), 3.40 (2H, t, —CH₂), 3.61 (4H, m, —CH₂), 3.67 (2H, m, —CH₂), 3.72-3.73 (4H, m, —CH₂); M⁺ + 1 found at 386.1. |
| | Oily compound | |
| 3 | 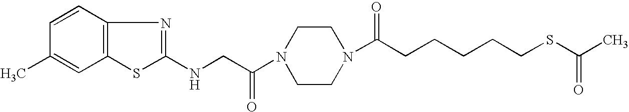 | ¹H NMR (400 MHz, DMSO): δ 1.28 (2H, m, —CH₂), 1.43 (2H, m, —CH₂), 2.32 (3H, s, —CH₃), 2.35 (3H, s, —CH₃), 2.86 (2H, t, —CH₂), 3.64 (8H, m, —CH₂), 4.99 (2H, m, —CH₂), 6.85 (1H, d, —CH), 6.96 (1H, d, —CH), 7.07 (1H, s, —CH); M⁺ + 1 found at 463.1. |
| | Yellow solid | |

### EXAMPLE 4

### Synthesis of 2-14-(5-Thioacetylpentanoyl)piperazin-1-yl]-N-(4-methyl-1,3-thiazol-2-yl)acetamide

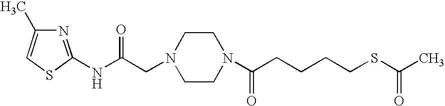 Step-I

### Synthesis of 2-Bromo-N-(4-methyl-1,3-thiazol-2-yl)acetamide

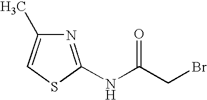

To a solution of 2-Amino-4-methyl thiazole (2 g, 17.51 mmol) in dichloromethane (20 ml) at 0-5° C. was added drop wise and simultaneously, bromoacetyl bromide (2.28 ml, 26.28 mmol) and triethylamine (0.2 ml). The reaction mixture was stirred for 1.5 hours, subsequently the solvent was stripped off and cold water was added. It was extracted twice with ethyl acetate; the combined organic layer was washed with brine, dried over anhydrous Na₂SO₄ and concentrated under reduced pressure to give 2-bromo-N-(4-methyl-1,3-thiazol-2-yl)acetamide (3.7 g, 90.01%).

Step-II

### Synthesis of 2-Iodo-N-(4-methyl-1,3-thiazol-2-yl)acetamide

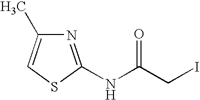 To a solution of 2-Bromo-N-(4-methyl-1,3-thiazol-2-yl)acetamide (0.5 g, 2.26 mmol) in acetone (20 ml) was added sodium iodide (0.67 g, 4.52 mmol), and the reaction mixture was stirred for 2 hours. Subsequently the solvent was stripped off and it was extracted twice with ethyl acetate, the combined organic layer was washed with brine, dried over anhydrous Na₂SO₄ and concentrated under reduced pressure to give 2-iodo-N-(4-methyl-1,3-thiazol-2-yl)acetamide (0.42 g, 70%).
Step-III

### Synthesis of 2-[4-(5-Thioacetylpentanoyl)piperazin-1-yl]-N-(4-methyl-1,3-thiazol-2-yl)acetamide

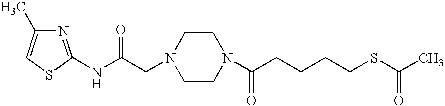

2-Iodo-N-(4-methyl-1,3-thiazol-2-yl)acetamide (0.4 g, 1.42 mmol) and S-{5-[4-(bromoacetyl)piperazin-1-yl]-5-oxopentyl}ethanethioate (0.35 g, 1.42 mmol) were taken up in acetone (20 ml) and triethylamine (0.2 ml) was added drop wise to it. The reaction mixture was stirred for 3 hours, subsequently the solvent was stripped off and it was extracted twice with ethyl acetate. The combined organic layer was washed with brine, dried over anhydrous Na₂SO₄ and concentrated under reduced pressure. The resulting crude material was purified by column chromatography using methanol and dichloromethane mixture to give pure 2-[4-(5-thioacetylpentanoyl)piperazin-1-yl]-N-(4-methyl-1,3-thiazol-2-yl)acetamide as an oily compound (0.2 g, 35.3%). ¹H NMR (400 MHz, CDCl₃): δ1.63-1.72 (4H, m, —CH₂), 2.33 (3H, s, —CH₃), 2.36 (3H, s, —CH₃), 2.58-2.62 (4H, m, —CH₂), 2.88 (2H, t, —CH₂), 3.27 (2H, s, —CH₂), 3.55 (2H, t, —CH₂), 3.71 (2H, t, —CH₂), 6.50 (1H, s, —CH), 10.25 (1H, brs, —NH); M⁺+1 found at 399.1.

The Following Compounds were Synthesized Using the Procedure Given in the Example 4.

| Ex. | Structure | Analytical Data |
|---|---|---|
| 5 | 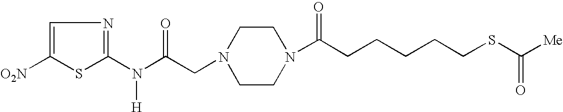 | ¹H NMR (400 MHz, CDCl₃): δ 1.59-1.66 (6H, m, —CH₂), 2.32 (3H, s, —CH₃), 2.33-2.36 (2H, t, —CH₂), 2.64 (4H, m, —CH₂), 2.87 (2H, t, —CH₂), 3.36 (2H, s, —CH₂), 3.58 (2H, t, —CH₂), 3.73 (2H, t, —CH₂), 8.33 (1H, s, —CH), 10.25 (1H, brs, —NH); M⁺ + 1 found at 444.1. |
| | Oily compound | |
| 6 | 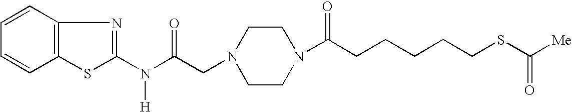 | ¹H NMR (400 MHz, CDCl₃): δ 1.4-1.42 (2H, m, —CH₂), 1.59-1.66 (4H, m, —CH₂), 2.33 (3H, s, —CH₃), 2.34-2.36 (2H, t, —CH₂), 2.63 (4H, m, —CH₂), 2.86 (2H, t, —CH₂), 3.34 (2H, s, —CH₂), 3.6 (2H, t, —CH₂), 3.75 (2H, t, —CH₂), 7.34 (1H, dd, —CH), 7.46 (1H, dd, —CH), 7.81 (2H, dd, |
| # —CH), 10.25 (1H, brs, —NH); M⁺ + 1 found at 449.1. | | |
| | Oily compound | |
| 7 | 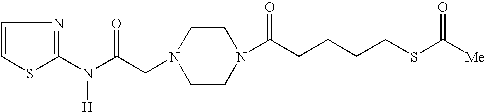 | ¹H NMR (400 MHz, CDCl₃): δ 1.61-1.66 (4H, m, —CH₂), 2.33 (3H, s, —CH₃), 2.36 (2H, t, —CH₂), 2.59-2.63 (4H, m, —CH₂), 2.90 (2H, t, —CH₂), 3.29 (2H, s, —CH₂), 3.56 (2H, t, —CH₂), 3.71 (2H, t, —CH₂), 7.0 (1H, d, —CH), 7.46-7.47 (1H, d, —CH), 10.25 (1H, brs, —NH); M⁺ + 1 found at 385.1. |
| | Oily compound | |
| 8 | 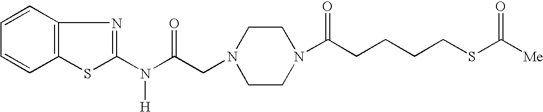 | ¹H NMR (400 MHz, CDCl₃): δ 1.61-1.73 (4H, m, —CH₂), 2.33 (3H, s, —CH₃), 2.36 (2H, t, —CH₂), 2.63—2.64 (4H, m, —CH₂), 2.90 (2H, t, —CH₂), 3.33 (2H, s, —CH₂), 3.59 (2H, t, —CH₂), 3.74 (2H, t, —CH₂), 7.34 (1H, dd, —CH), 7.47 (1H, dd, —CH), 7.82 (2H, dd, —CH), 10.25 (1H, brs, —NH); M⁺ + 1 found at 435.1. |
| | Oily compound | |
| 9 | 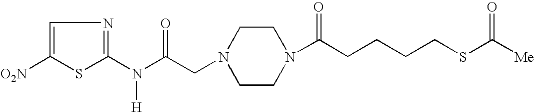 | ¹H NMR (400 MHz, CDCl₃): δ 1.61-1.72 (4H, m, —CH₂), 2.33 (3H, s, —CH₃), 2.34-2.37 (2H, t, —CH₂), 2.62 (4H, m, —CH₂), 2.89 (2H, t, —CH₂), 3.36 (2H, s, —CH₂), 3.58 (2H, t, —CH₂), 3.73 (2H, t, —CH₂), 8.33 (1H, s, —CH), 10.27 (1H, brs, —NH); M⁺ + 1 found at 430.1. |
| | Low melting solid | |
| 10 | 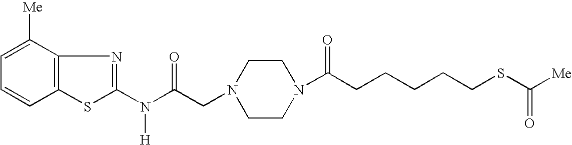 | ¹H NMR (400 MHz, CDCl₃): δ 1.43-1.44 (2H, m, —CH₂), 1.59-1.67 (4H, m, —CH₂), 2.33 (3H, s, —CH₃), 2.34-2.36 (2H, t, —CH₂), 2.63-2.64 (4H, m, —CH₂), 2.66 (3H, s, —CH₃), 2.86 (2H, t, —CH₂), 3.32 (2H, s, —CH₂), 3.60 (2H, t, —CH₂), 3.76 (2H, t, —CH₂), 7.21-7.26 (2H, m, —CH), 7.66 (1H, dd, —CH), 10.28 |
| # (1H, brs, —NH); M⁺ + 1 found at 463.1; m.p: 122-124° C. | | |
| | Crème colored solid | |
| 11 | 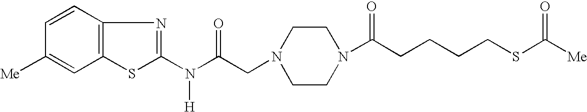 | ¹H NMR (400 MHz, CDCl₃): δ 1.64-1.72 (4H, m, —CH₂), 2.32 (3H, s, —CH₃), 2.35 (2H, t, —CH₂), 2.48 (3H, s, —CH₃), 2.63 (4H, t, —CH₂), 2.89 (2H, t, —CH₂), 3.30 (2H, s, —CH₂), 3.58 (2H, t, —CH₂), 3.73 (2H, t, —CH₂), 7.27 (2H, dd, —CH), 7.66 (1H, dd, —CH), 10.29 (1H, brs, —NH); M⁺ + 1 found at 449.3; m.p: |
| # 119-122° C. | | |
| | Pale brown colored solid | |
| 12 | 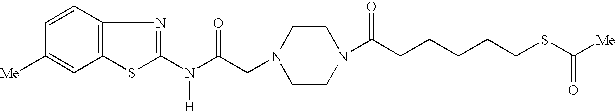 | ¹H NMR (400 MHz, CDCl₃): δ 1.44-1.68 (6H, m, —CH₂), 2.32 (3H, s, —CH₃), 2.32-2.35 (2H, t, —CH₂), 2.48 (3H, s, —CH₃), 2.63 (4H, t, —CH₂), 2.86-2.89 (2H, t, —CH₂), 3.32 (2H, s, —CH₂), 3.73 (2H, t, —CH₂), 4.41 (2H, t, —CH₂), 7.27 (1H, d, —CH), 7.62-7.69 (2H, dd, —CH), 10.32 (1H, brs, —NH); M⁺ + 1 found |
| # at 463.1; m.p: 127-131° C. | | |
| | Crème colored solid | |
| 13 | 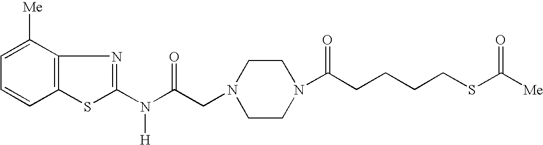 | ¹H NMR (400 MHz, CDCl₃): δ 1.64-1.73 (4H, m, —CH₂), 2.33 (3H, s, —CH₃), 2.36 (2H, t, —CH₂), 2.63 (4H, t, —CH₂), 2.65 (3H, s, —CH₃), 2.89 (2H, t, —CH₂), 3.32 (2H, s, —CH₂), 3.70 (2H, t, —CH₂), 7.24 (2H, m, —CH), 7.65 (1H, d, —CH), 10.20 (1H, brs, —NH); M⁺ + 1 found at 449.1; m.p: 108-112° C. |
| | Crème colored solid | |
| 14 | 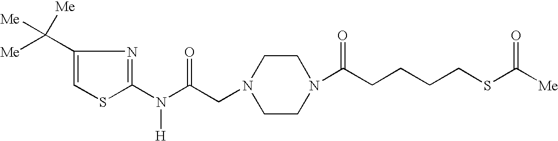 | ¹H NMR (400 Mhz, CDCl₃): δ 1.31-1.66 (9H, s, —CH₃), 1.65 (4H, m, —CH₂), 2.33 (3H, s, —CH₃), 2.35 (2H, t, —CH₂), 2.60 (4H, t, —CH₂), 2.88 (2H, t, —CH₂), 3.26 (2H, s, —CH₂), 3.59 (2H, t, —CH₂), 3.74 (2H, t, —CH₂), 6.57 (1H, s, —CH), 10.1 (1H, brs, —NH); M⁺ + 1 found at 441.0; m.p: 85-88° C. |
| | White powder | |
| 15 | 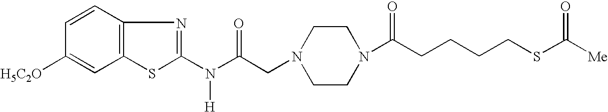 | ¹H NMR (400 MHz, CDCl₃): δ 1.46 (3H, t, —CH₃), 1.69 (4H, m, —CH₂), 2.33 (3H, s, —CH₃), 2.36 (2H, t, —CH₂), 2.63 (4H, t, —CH₂), 2.89 (2H, t, —CH₂), 3.31 (2H, s, —CH₂), 3.57 (2H, t, —CH₂), 3.72 (2H, t, —CH₂), 4.10 (2H, t, —CH₂), 7.04 (1H, d, —CH), 7.27 (1H, d, —CH), 7.67 (1H, d, —CH), 10.27 (1H, brs, —NH); |
| # M⁺ + 1 found at 478.8; m.p: 115-120° C. | | |
| | Crème colored solid | |
| 16 | 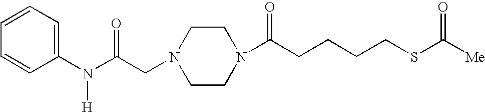 | ¹H NMR (400 MHz, CDCl₃): δ 1.67 (4H, m, —CH₂), 2.33 (3H, s, —CH₃), 2.36 (2H, t, —CH₂), 2.62 (4H, t, —CH₂), 2.89 (2H, t, —CH₂), 3.18 (2H, s, —CH₂), 3.55 (2H, t, —CH₂), 3.71 (2H, t, —CH₂), 4.10 (2H, t, —CH₂), 7.13 (1H, d, —CH), 7.35 (2H, d, —CH), 7.55 (2H, d, —CH), 8.97 (1H, brs, —NH); M⁺ + 1: 378.1. |
| | Oily compound | |
| 17 | 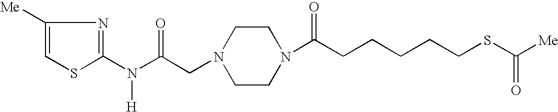 | ¹H NMR (400 MHz, CDCl₃): δ 1.42-1.66 (6H, m, —CH₂), 2.31 (3H, s, —CH₃), 2.33 (2H, t, —CH₂), 2.37 (3H, s, —CH₃), 2.59 (4H, m, —CH₂), 2.87 (2H, t, —CH₂), 3.27 (2H, s, —CH₂), 3.55 (2H, t, —CH₂), 3.70 (2H, t, —CH₂), 6.56 (1H, s, —CH), 10.13 (1H, brs, —NH); M⁺ + 1 found at 413.1; m.p: 70-73° C. |
| | Crème colored solid | |
| 18 | 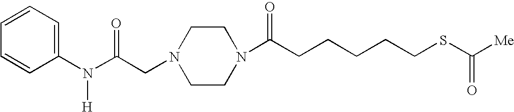 | ¹H NMR (400 MHz, CDCl₃): δ 1.42 (4H, m, —CH₂), 1.57-1.68 (4H, m, —CH₂), 2.32 (3H, s, —CH₃), 2.35 (2H, t, —CH₂), 2.62 (4H, t, —CH₂), 2.85 (2H, t, —CH₂), 2.89 (2H, s, —CH₂), 3.51 (2H, t, —CH₂), 3.71 (2H, t, —CH₂), 7.13 (1H, dd, —CH), 7.35 (2H, d, —CH), 7.52-7.57 (2H, |
| # d, —CH), 8.97 (1H, brs, —NH); M⁺ + 1 found at 392.2. | | |
| | Oily compound | |
| 19 | 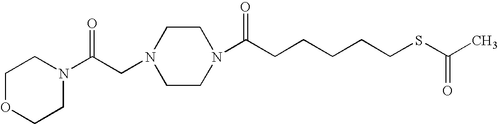 | ¹H NMR (400 MHz, CDCl₃): δ 1.42 (2H, m, —CH₂), 1.56-1.68 (6H, m, —CH₂), 2.3 (2H, t, —CH₂), 2.32 (3H, s, —CH₃), 2.48-2.52 (4H, m, —CH₂), 2.85-2.88 (2H, t, —CH₂), 3.21 (2H, s, —CH₂), 3.48 (2H, t, —CH₂), 3.60-3.68 (8H, m, —CH₂); M⁺ + 1 found at 386.2. |
| | Oily compound | |
| 20 | 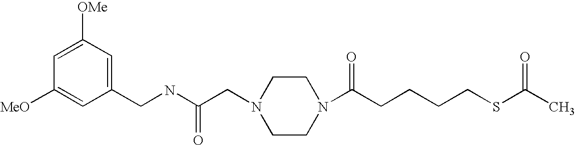 | ¹H NMR (400 MHz, CDCl₃): δ 1.60-1.71 (4H, m, —CH₂), 2.30 (3H, s, —CH₃), 2.33 (2H, t, —CH₂), 2.46 (4H, t, —CH₂), 2.88 (2H, t, —CH₂), 3.02 (2H, s, —CH₂), 3.44 (2H, t, —CH₂), 3.58 (2H, t, —CH₂), 3.81 (3H, s, —CH₃), 3.84 (3H, s, —CH₃), 4.39 (2H, t, —CH₂), 6.42-6.47 (1H, d, —CH), 7.17 (1H, d, —CH), 7.47 |
| # (1H, d, —CH); M⁺ + 1 found at 452.2. | | |
| | Oily compound | |
| 21 | 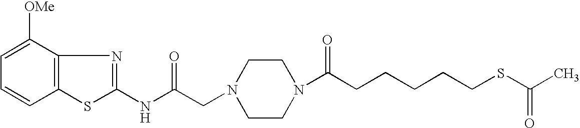 | ¹H NMR (400 MHz, CDCl₃): δ 1.43 (2H, m, —CH₂), 1.58-1.66 (4H, m, —CH₂), 2.32 (3H, s, —CH₃), 2.60 (4H, t, —CH₂), 2.75 (2H, t, —CH₂), 2.88 (2H, t, —CH₂), 3.71 (2H, t, —CH₂), 4.02 (3H, s, —CH₃), 6.91 (1H, d, —CH), 7.26 (1H, d, —CH), 7.42 (1H, d, —CH), 10.45 (1H, brs, —NH); M⁺ + 1 found at 479.1. |
| | Crème colored solid | |
| 22 | 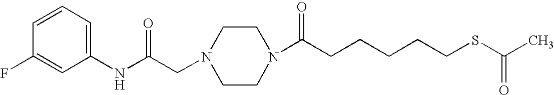 | ¹H NMR (400 MHz, CDCl₃): δ 1.43 (2H, m, —CH₂), 1.58-1.68 (4H, m, —CH₂), 2.32 (3H, s, —CH₃), 2.35 (2H, t, —CH₂), 2.62 (4H, t, —CH₂), 2.87 (2H, t, —CH₂), 3.18 (2H, s, —CH₂), 3.56 (2H, t, —CH₂), 3.71 (2H, t, —CH₂), 6.83 (1H, m, —CH), 7.20 (1H, d, —CH), 7.28 (1H, d, —CH), 7.54 (1H, d, —CH), 9.04 (1H, |
| # brs, —NH); M⁺ + 1 found at 410.1. | | |
| | Oily compound | |
| 23 | 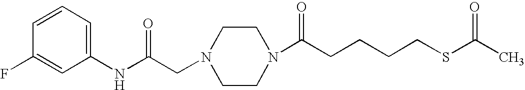 | ¹H NMR (400 MLHz, CDCl₃): δ 1.57-1.72 (4H, m, —CH₂), 2.32 (3H, s, —CH₃), 2.36 (2H, t, —CH₂), 2.61 (4H, t, —CH₂), 2.89 (2H, t, —CH₂), 3.18 (2H, s, —CH₂), 3.56 (2H, t, —CH₂), 3.70 (2H, t, —CH₂), 6.80 (1H, d, —CH), 7.20 (1H, s, —CH), 7.28 (1H, m, —CH), 7.54 (1H, d, —CH), 9.10 (1H, brs, —NH); M⁺ + 1 |
| # found at 396.1. | | |
| | Oily compound | |

### EXAMPLE 24

### Synthesis of N-1,3-benzothiazol-2-yl-2-[4-(6-mercaptohexanoyl)piperazin-1-yl]acetamide

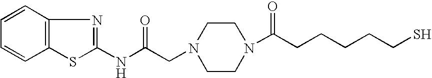

N-1,3-benzothiazol-2-ylamino-2-[4-(5-thioacetylpentanoyl)piperazin-1-yl]acetamide (0.2 g, 0.46 mmol) was dissolved in ethanol (5 ml) and tetrahydrofuran (5 ml) added 2N aqueous NaOH (10 ml), stirred for 3 hours. Diluted with ethyl acetate (100 ml) and separated the organic layer. The combined organic layer was washed with brine, dried over anhydrous Na₂SO₄ and concentrated under reduced pressure. The resulting crude material was purified by column chromatography using methanol and dichloromethane mixture to give pure N-1,3-benzothiazol-2-yl-2-[4-(6-mercaptohexanoyl)piperazin-1-yl]acetamide as an amorphous powder (0.06 g, 33.15%). ¹H NMR (400 MHz, CDCl₃): δ1.47 (2H, m, —CH₂), 1.65-1.74 (4H, m, —CH₂), 2.35 (2H, t, —CH₂), 2.63-2.71 (6H, m, —CH₂), 3.33 (2H, s, —CH₂), 3.59 (2H, t, —CH₂), 3.74 (2H, t, —CH₂), 7.32-7.36 (1H, m, —CH), 7.44-7.48 (1H, m, —CH), 7.79-7.85 (2H, dd, —CH), 10.25 (1H, brs, —NH); m.p: 89-92° C.

The Following Compounds Were Synthesized Using the Procedure Given in the Example 24.

| Ex. | Structure | Analytical Data |
|---|---|---|
| 25 | 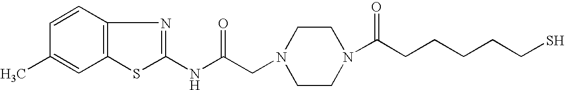 | ¹H NMR (400 MHz, CDCl₃): δ 1.46 (2H, m, -CH₂), 1.65-1.74 (6H, m, —CH₂), 2.35 (2H, t, —CH₂), 2.48 (3H, s, —CH₃), 2.64 (2H, t, —CH₂), 2.67-2.71 (4H, m, —CH₂), 3.32 (2H, s, —CH₂), 3.58 (2H, t, —CH₂), 3.73 (2H, t, —CH₂), 7.27 (1H, d, —CH), 7.62 (1H, s, —CH), 7.67 (1H, d, —CH); m.p: 213-216° C. |
| | Crème colored solid | |

### EXAMPLE 26

### Synthesis of N-1,3-benzothiazol-2-yl-2-{6-[6(hydroxyamino)hexanoyl]piperazin-1-yl}acetamide

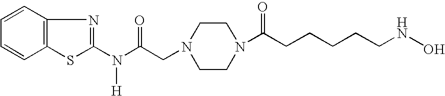 Step-I

### Synthesis of 1-(6-Bromohexanoyl)piperazine

TFA (1.01 ml, 13.21 mmol) was added to t-Butyl 4-(6-bromohexanoyl)piperazine-1-carboxylate (0.8 g, 2.2 mmol), and the reaction mixture was stirred for 3 hours. Subsequently the reaction mixture was diluted with ethyl acetate; NaHCO₃ (4.39 g, 52.3 mmol) was added and it was stirred for another 1 hour, and then filtered through a Hirsch funnel. The resulting filtrate was concentrated under reduced pressure, and was used as such for the next reaction. 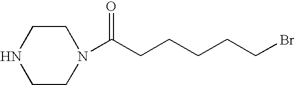
Step-II

### Synthesis of N-1,3-benzothiazol-2-yl-2-bromoacetamide

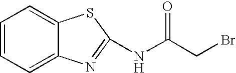

To a solution of 2-Amino-benzothiazole (2 g, 13.3 mmol) in dichloromethane (20 ml) at 0-5° C. was added drop wise, bromoacetyl bromide (1.74 ml, 19.97 mmol) and triethylamine (3.7 ml, 26.6 mmol) in dichloromethane (each 2 ml) simultaneously. The reaction mixture was stirred for 1.5 hours, subsequently the solvent was stripped off and cold water was added. It was extracted with ethyl acetate twice; the combined organic layer was washed with brine, dried over anhydrous Na₂SO₄ and concentrated under reduced pressure to give pure N-1,3-benzothiazol-2-yl-2-bromoacetamide (3.1 g, 86.11%).

Step-III

### Synthesis of N-1,3-benzothiazol-2-yl-2-[6-(6-bromohexanoyl)piperazin-1-yl]acetamide

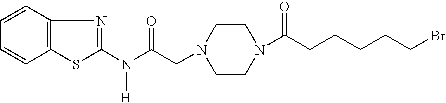

N-1,3-benzothiazol-2-yl-2-bromoacetamide (1.55 g, 5.70 mmol) and sodium iodide (0.427 g, 2.85 mmol) were in to acetone, stirred for 2 hour. Added K₂CO₃ (0.157 g, 1.14 mmol), 1-(6-bromohexanoyl)piperazine (1.50 g, 5.7 mmol) followed by triethyl amine (28.5 mmol) drop wise and continued stirring for another 2 hours. Subsequently the solvent was stripped off and it was extracted twice with ethyl acetate. The combined organic layer was washed with brine, dried over anhydrous Na₂SO₄ and concentrated under reduced pressure. The resulting extract was washed with 10% ethylacetate in hexane (15 ml×2) to give pure N-1,3-benzothiazol-2-yl-2-[6-(6-bromohexanoyl)piperazin-1-yl]acetamide (0.25 g, 9.7%).

Step-IV

### N-1,3-benzothiazol-2-yl-2-{6-[6 (hydroxyamino)hexanoyl]piperazin-1-yl}acetamide

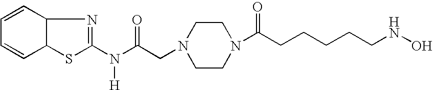

N-1,3-benzothiazol-2-yl-2-[6-(6-bromohexanoyl)piperazin-1-yl]acetamide (0.25 g, 0.55 mmol) was dissolved in DMSO (20 ml), added hydroxalamine hydrochloride (0.16 g, 1.66 mmol) followed by triethyl amine (0.4 ml, 2.76 mmol). Stirred the reaction for 4 hours, added water (75 ml) and extracted twice with ethylacetate (150 ml). The combined organic layer was washed with brine, dried over anhydrous Na₂SO₄ and concentrated under reduced pressure. The resulting crude material was purified by column chromatography using methanol and dichloromethane mixture to give pure N-1,3-benzothiazol-2-yl-2-{6-[6(hydroxyamino)hexanoyl]piperazin-1-yl}acetamide as an crème coloured amorphous powder (0.08 g, 37.8%). ¹H NMR (400 MHz, CDCl₃): 61.49 (2H, m, —CH₂), 1.68 (2H, m, —CH₂), 1.82 (2H, m, —CH₂), 2.36 (2H, m, —CH₂), 2.63 (4H, m, —CH₂), 3.33 (2H, s, —CH₂), 3.54-3.59 (4H, m, —CH₂), 3.73 (2H, t, —CH₂), 7.34 (1H, dd, —CH), 7.46 (1H, dd, —CH), 7.81 (2H, dd, —CH), 10.4 (1H, brs, —NH); m.p: 150-152° C.

The Following Compounds Were Synthesized Using the Procedure Given in the Example 26.

| Ex. | Structure | Analytical Data |
|---|---|---|
| 27 | 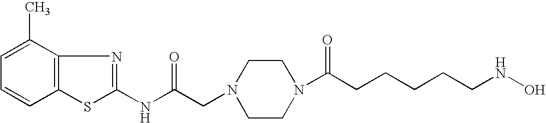 | ¹H NMR (400 MHz, CDCl₃): δ 1.51 (2H, m, —CH₂), 1.69 (2H, m, —CH₂), 1.81 (2H, m, —CH₂), 2.17 (2H, t, —CH₂), 2.65 (4H, m, —CH₂), 2.66 (3H, s, —CH₃), 3.56 (2H, t, —CH₂), 3.62 (2H, t, —CH₂), 3.77 (2H, t, —CH₂), 7.23 (1H, d, —CH), 7.66 (1H, d, —CH), 10.23 (1H, brs, —NH); m.p: 137-140° C. |
| | Crème colored solid | |
| 28 | 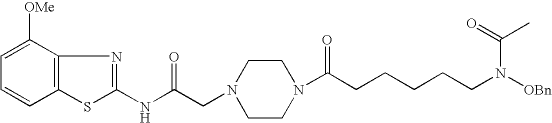 | ¹H NMR (400 MHz, CDCl₃): δ 1.36 (4H, m, —CH₂), 1.65 (3H, s, —CH₃), 1.67 (2H, m, —CH₂), 2.32 (2H, t, —CH₂), 2.60 (4H, m, —CH₂), 3.30 (2H, s, —CH₂), 3.65 (2H, m, —CH₂), 3.70 (4H, m, —CH₂), 4.04 (3H, s, —CH₃), 4.82 (2H, s, —CH₂), 6.91 (1H, d, —CH), 7.29 (1H, d, —CH), 7.41 (6H, m, —CH), 10.40 (1H, brs, —NH); |
| # M⁺ + 1 found at 568.2. | | |
| | Oily compound | |

### EXAMPLE 29

### Synthesis of 8-{4-[2-(1,3-Benzothiazol-2-ylamino)-2-oxoethyl]piperazin-1-yl}-N-hydroxy-8-oxo octanamide

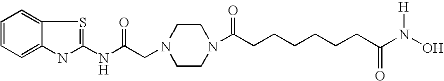 Step-I

### Synthesis of N-1,3-benzothiazol-2-yl-2-bromoacetamide

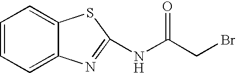

To a solution of 2-Amino-benzothiazole (2 g, 13.3 mmol) in dichloromethane (20 ml) at 0-5° C. was added drop wise, bromoacetyl bromide (1.74 ml, 19.97 mmol) and triethylamine (3.7 ml, 26.6 mmol) in dichloromethane (each 2 ml) simultaneously. The reaction mixture was stirred for 1.5 hours, subsequently the solvent was stripped off and cold water was added. It was extracted with ethyl acetate twice; the combined organic layer was washed with brine, dried over anhydrous Na₂SO₄ and concentrated under reduced pressure to give pure N-1,3-benzothiazol-2-yl-2-bromoacetamide (3.1 g, 86.11%).

Step-II

### Synthesis of t-Butyl-4-[2-(1,3-benzothiazol-2-ylamino)-2-oxoethyl]piperazine-1-carboxylate

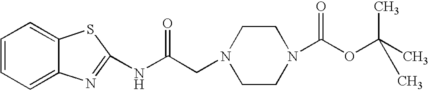

To a solution of N-1,3-benzothiazol-2-yl-2-bromoacetamide (1.75 g, 6.44 mmol) in THF (25 ml) was added tert-butyl piperazine-1-carboxylate (1 g, 53.69 mmol) and K₂CO₃ (1.48 g, 10.73 mmol), and the reaction mixture was stirred for 15 hours. Subsequently the solvent was stripped off completely and cold water was added. It was extracted twice with ethyl acetate; the combined organic layer was washed with brine, dried over anhydrous Na₂SO₄ and concentrated under reduced pressure to give crude t-Butyl 4-[2-(1,3-benzothiazol-2-ylamino)-2-oxoethyl]piperazine-1-carboxylate (1.7 g, 85%).

Step-III

### Synthesis of N-1,3-benzothiazol-2-yl-2-piperazin-1-yl acetamide

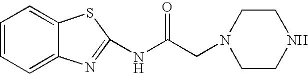

The above t-Butyl 4-[2-(1,3-benzothiazol-2-ylamino)-2-oxoethyl]piperazine-1-carboxylate (1.5 g, 3.99 mmol) was stirred with TFA (3.06 ml, 39.9 mmol) for 5 hours. Later the reaction mixture was diluted with ethyl acetate, and 2.5 g of sodium bicarbonate was added, and stirred for 0.5 hours. Subsequently the reaction mixture was filtered and the filtrate was concentrated under reduced pressure to give N-1,3-benzothiazol-2-yl-2-piperazin-1-yl acetamide.

Step-IV

### Synthesis of 8-{4-[2-(1,3-Benzothiazol-2-ylamino)-2-oxoethyl]piperazin-1-yl}-8-oxo octanoic acid

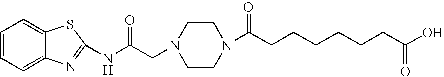

To a solution of N-1,3-benzothiazol-2-yl-2-piperazin-1-yl acetamide (1 g, 3.62 mmol) in THF (25 ml) was added suberic acid (0.76 g, 4.35 mmol), benzotriazol-1-ylozy-tris (dimethylamino)-phosphonium hexafluorophosphate (BOP) (1.92 g, 4.35 mmol), and 1-hydroxybenzotriazole (HOBt) (0.59 g, 4.35 mmol), and the reaction mixture was stirred for 15 minutes. N,N-diisopropylethyleneamine (DIPEA) (0.76 ml, 4.35 mmol) was added drop wise to the above, and the stirring was continued for 15 hours. Subsequently the THF was stripped off, and water was added to the reaction mixture. It was extracted twice with ethyl acetate, and the combined organic layer was washed with brine, dried over anhydrous Na₂SO₄ and concentrated under reduced pressure. 50 ml of 5% aqueous NaOH solution was added to the resulting crude and it was stirred for half an hour, subsequently it was extracted twice with ethyl acetate. The aqueous layer was slowly acidified with concentrated HCl till pH 3-4, extracted thrice with ethyl acetate; the combined organic layer was washed with brine, dried over anhydrous Na₂SO₄ and concentrated under reduced pressure to give pure 8-{4-[2-(1,3-benzothiazol-2-ylamino)-2-oxoethyl]piperazin-1-yl}-8-oxo octanoic acid (1.1 g, 70.3%).

Step-V

### Synthesis of 8-{4-[2-(1,3-Benzothiazol-2-ylamino)-2-oxoethyl]piperazin-1-yl}-N-hydroxy-8-oxo octanamide

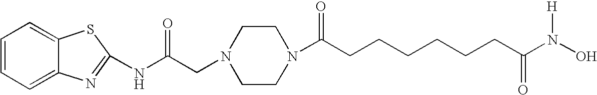

To the above acid (1 g, 2.32 mmol) in THF (20 ml) was added hydroxylamine hydrochloride (0.26 g, 3.49 mmol), Benzotriazol-1-ylozy-tris(dimethylamino)-phosphonium hexafluorophosphate (BOP) (1.23 g, 2.78 mmol), and 1-hydroxybenzotriazole (HOBt) (0.37 g, 2.78 mmol), and the reaction mixture was stirred for 15 minutes. N,N-diisopropylethyleneamine (DIPEA) (1.18 ml, 6.95 mmol) was added drop wise to the above, and the stirring was continued for 8 hours. Subsequently the THF was stripped off and water was added to the reaction mixture. It was extracted thrice with ethyl acetate. The combined organic layer was washed with brine, dried over anhydrous Na₂SO₄ and concentrated under reduced pressure. The resulting concentrate was purified by preparative HPLC using methanol and acetonitrile mixture to give pure 8-{4-[2-(1,3-Benzothiazol-2-ylamino)-2-oxoethyl]piperazin-1-yl}-N-hydroxy-8-oxo octanamide as a colorless solid (0.04 g, 3.9%). ¹H NMR (400 MHz, CDCl₃): δ1.22-1.36 (4H, m, —CH₂), 1.64 (4H, m, —CH₂), 2.18 (2H, t, —CH₂), 2.36 (2H, t, —CH₂), 2.64 (4H, m, —CH₂), 3.35 (2H, s, —CH₂), 3.61 (2H, m, —CH₂), 3.75 (2H, m, —CH₂), 7.34 (1H, dd, —CH), 7.47 (1H, dd, —CH), 7.82 (2H, dd, —CH); M⁺+1 found at 448.1; m.p: 166-170° C.

The Following Compounds were Synthesized Using the Procedure Given in the Example 29

| Exam- | | |
|---|---|---|
| ple | Structure | Analytical Data |
| 30 | 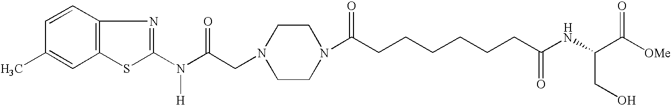 | ¹H NMR (400 MHz, CDCl₃): δ 1.39 (4H, m, —CH₂), 1.66 (4H, m, —CH₂), 2.28 (2H, t, —CH₂), 2.35 (2H, t, —CH₂), 2.48 (3H, s, —CH₃), 2.63 (4H, t, —CH₂), 3.32 (2H, s, —CH₂), 3.59 (2H, t, —CH₂), 3.72 (2H, t, —CH₂), 3.78 (3H, s, —CH₃), 3.90 (2H, m, —CH₂), 4.70 (1H, t, —CH), 7.26 (1H, s, —CH), 7.57 (1H, d, |
| # —CH), 7.67 (1H, d, —CH), 10.31 (1H, brs, —NH); M⁺ + 1 found at 548.2. | | |
| | Oily compound | |

### EXAMPLE 31

### Synthesis of 8-{4-[2-(Phenyl-2-ylamino)-2-oxoethyl]piperazin-1-yl}-N-hydroxy-8-oxo octanamide

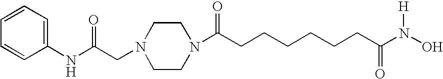 Step-I

### Synthesis of ethyl-8-(4-Benzylpiperazin-1-yl)-8-oxo octanoate

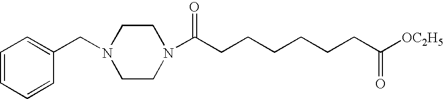

Benzotriazol-1-ylozy-tris(dimethylamino)-phosphonium hexafluorophosphate (BOP) (10.50 g, 23.73 mmol), 1-hydroxybenzotriazole (HOBt) (3.2 g, 23.73 mmol), 1-benzylpiperazine. (4.18, 23.73 mmol) and ethyl hydrogen suberate (4 g, 19.78 mmol) were all taken up in THF (100 ml) and stirred for ½ hour. N,N-diisopropylethyleneamine (DIPEA) (10.32 ml, 59.34 mmol) was added drop wise to the above and stirring was continued for 6 hours. Subsequently the THF was stripped off and water was added to the reaction mixture. It was extracted thrice with dichloromethane; the combined organic layer was washed with brine, dried over anhydrous Na₂SO₄ and concentrated under reduced pressure. The resulting crude material was purified by column chromatography using ethylacetate and n-hexane mixture to give pure ethyl-8-(4-benzylpiperazin-1-yl)-8-oxo octanoate (5.1 g, 71.63%).

Step-II

### Synthesis of Ethyl 8-oxo-8-piperazin-1-yloctanoate

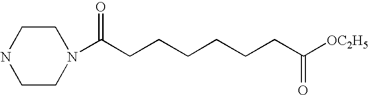

Ethyl-8-(4-benzylpiperazin-1-yl)-8-oxo octanoate (5 g, 13.89 mmol) was dissolved in methanol (75 ml), added 10% of palladium on carbon (1 g, 20%) and stirred under hydrogen atmosphere for 4 hrs. Filter the reaction mixture through celite bed and concentrated the filtrate under reduced pressure to get pure ethyl 8-oxo-8-piperazin-1-yloctanoate (3.2 g, 85.65%).

Step-III

### 8-{4-[2-(Phenyl-2-ylamino)-2-oxoethyl]piperazin-1-yl}-8-oxooctanoate

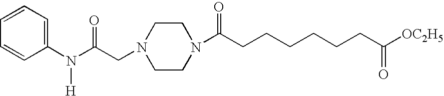

2-Bromo-N-phenylacetamide (0.29 g, 1.33 mmol) and sodium iodide (NaI) (0.083 g, 0.55 mmol) were stirred for ½ hour, added Potassium carbonate (K₂CO₃) (0.08 g, 0.55 mmol) and ethyl-8-oxo-8-piperazin-1-yloctanoate (0.30 g, 1.11 mmol). Triethylamine (0.15 ml, 1.11 mmol) was added drop wise to it, stirred the reaction mixture for 2 hours, subsequently the solvent was stripped off and it was extracted twice with ethyl acetate. The combined organic layer was washed with brine, dried over anhydrous Na₂SO₄ and concentrated under reduced pressure to give 8-{4-[2-(phenyl-2-ylamino)-2-oxoethyl]piperazin-1-yl}-8-oxooctanoate (0.32 g, 71.27%).

Step-IV

### 8-{4-[2-(Phenyl-2-ylamino)-2-oxoethyl]piperazin-1-yl}-N-hydroxy-8-oxo octanamide

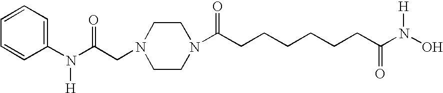

Hydroxalamine hydrochloride (0.93 g, 13.38 mmol), was taken into methanol (10 ml), cooled to 0-5° C., in another flask potassium hydroxide (KOH) (0.75 g, 13.38 mmol) dissolved in methanol (10 ml) and cooled to 0-5° C. Added alcoholic KOH solution to the above amine solution, filtered through filter paper under cooling condition. Added the filtrate to the mixture of 8-{4-[2-(phenyl-2-ylamino)-2-oxoethyl]piperazin-1-yl}-8-oxooctanoate (0.3 g, 0.74 mmol), potassium hydroxide (0.13 g, 2.2 mmol). Added 10 ml of methanol and stirred the reaction for 5 hrs. Added ice and adjusted the pH to 7 using glacial acetic acid. Extracted twice with ethyl acetate. The combined organic layer was washed with brine, dried over anhydrous Na₂SO₄ and concentrated under reduced pressure. The resulting crude material was purified by column chromatography using methanol and dichloromethane mixture to give pure crème coloured solid 8-{4-[2-(phenyl-2-ylamino)-2-oxoethyl]piperazin-1-yl}-N-hydroxy-8-oxo octanamide (0.16 g, 57.15%). ¹H NMR (400 MHz, CDCl₃): δ1.37 (4H, m, —CH₂), 1.64 (4H, m, —CH₂), 2.16 (2H, t, —CH₂), 2.35 (2H, t, —CH₂), 2.63 (4H, m, —CH₂), 3.20 (2H, s, —CH₂), 3.56 (2H, t, —CH₂), 3.71 (2H, t, —CH₂), 7.13 (1H, dd, —CH), 7.35 (2H, dd, —CH), 7.56 (2H, dd, —CH), 8.50 (1H, brs, —OH), 9.01 (1H, brs, —NH), 9.25 (1H, brs, —NH); M⁺+1 found at 391.1; m.p: 138-140° C.

The Following Compounds were Synthesized Using the Procedure Given in the Example 31

| Example | Structure | Analytical Data |
|---|---|---|
| 32 | 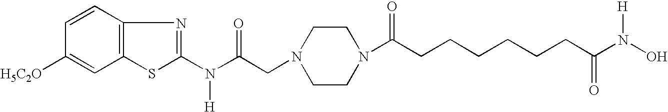 | ¹H NMR (400 MHz, DMSO): δ 1.24 (6H, m, —CH₂), 1.35 (3H, t, —CH₃), 1.46 (4H, m, —CH₂), 1.92 (2H, m, —CH₂), 1.99 (2H, m, —CH₂), 3.35 (4H, m, —CH₂), 3.49 (2H, t, —CH₂), 4.06 (2H, q, —CH₂), 7.01 (1H, d, —CH), 7.55 (1H, s, —CH), 7.62 (1H, d, —CH), 8.66 (1H, brs, —OH), 10.33 (1H, brs, —NH), 12.0 (1H, brs, |
| # —NH); M⁺ + 1 found at 491.8; m.p: 74-78° C. | | |
| | Crème coloured solid | |
| 33 | 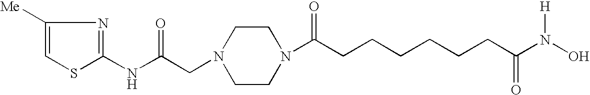 | ¹H NMR (400 MHz, CDCl₃): δ 1.35 (4H, m, —CH₂), 1.68 (4H, m, —CH₂), 2.18 (2H, t, —CH₂), 2.35 (2H, t, —CH₂), 2.37 (3H, s, —CH₃), 2.61 (4H, m, —CH₂), 3.30 (2H, s, —CH₂), 3.58 (2H, t, —CH₂), 3.72 (2H, t, —CH₂), 6.55 (1H, s, —CH), 9.30 (1H, brs, —NH), 10.6 (1H, brs, —NH); M⁺ + 1 found at 412.1. |
| | Crème coloured solid, | |
| | hygroscopic | |
| 34 | 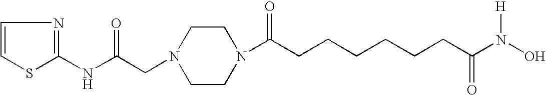 | ¹H NMR (400 MHz, CDCl₃): δ 1.28 (4H, m, —CH₂), 1.65 (4M, m, —CH₂), 2.18 (2H, t, —CH₂), 2.35 (2H, t, —CH₂), 2.62 (4H, m, —CH₂), 3.34 (2H, s, —CH₂), 3.59 (2H, t, —CH₂), 3.73 (2H, t, —CH₂), 7.01 (1H, d, —CH), 7.44 (1H, d, —CH), 9.50 (1H, brs, —NH), 10.6 (1H, brs, —NM); M⁺ + 1 found at 398.1. |
| | Crème coloured solid, | |
| | hygroscopic | |
| 35 | 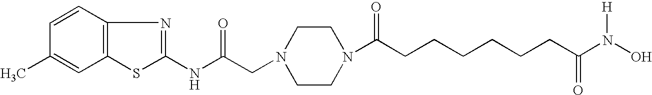 | ¹H NMR (400 MHz, DMSO): δ 1.25 (4H, m, —CH₂), 1.45 (4H, m, —CH₂), 1.93 (2H, t, —CH₂), 2.28 (2H, t, —CH₂), 2.41 (3H, s, —CH₃), 2.50 (4H, m, —CH₂), 3.34 (2H, s, —CH₂), 3.47 (4H, t, —CH₂), 7.25 (1H, d, —CH), 7.62 (1H, d, —CH), 7.77 (1H, s, —CH), 8.70 (1H, brs, —OH), 10.33 (1H, brs, —NH), 12.2 (1H, brs, |
| # —NH); M⁺ + 1 found at 462.1; m.p: 173-178° C. | | |
| | Pale yeallow powder | |
| 36 | 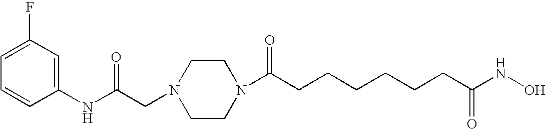 | ¹H NMR (400 MHz, CDCl₃): δ 1.25 (4H, m, —CH₂), 1.46 (4H, m, —CH₂), 1.92 (2H, t, —CH₂), 2.27 (2H, t, —CH₂), 2.45 (2H, t, —CH₂), 3.17 (2H, s, —CH₂), 3.49 (4H, m, —CH₂), 5.76 (2H, s, —CH₂), 6.89 (1H, m, —CH), 7.38 (2H, m, —CH), 8.65 (1H, d, —CH); M⁺ + 1 found at 409.1; m.p: 135-138° C. |
| | Crème coloured solid | |

### EXAMPLE 37

### Synthesis of 8-{4-[2-(6-Ethoxy-1,3-benzothiazol-2-ylamino)-2-oxoethyl]piperazin-1-yl}-N-acetyl-N-hydroxy-8-oxooctanamide

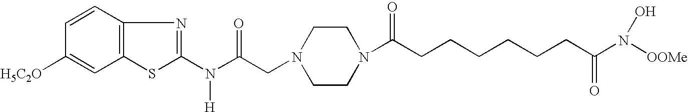

8-{4-[2-(6-Ethoxy-1,3-benzothiazol-2-ylamino)-2-oxoethyl]piperazin-1-yl}-N-hydroxy-8-oxo octanamide (0.12 g, 0.24 mmol) was dissolved in dichloromethane, added pyridine (.06 ml, 0.73 mmol), dimethylaminopyridine (0.003 g, 0.02 mmol) and stirred for 15 minutes, cooled the reaction temperature to 0-5° C. Added acetyl chloride (0.08 ml, 0.73 mmol) and continued stirring for 4 hrs. Added water (50 ml) and extracted twice with dichloromethane (100 ml). The combined organic layer was washed with brine, dried over anhydrous Na₂SO₄ and concentrated under reduced pressure. The resulting crude material was purified by column chromatography using methanol and dichloromethane mixture to give pure 8-{4-[2-(6-ethoxy-1,3-benzothiazol-2-ylamino)-2-oxoethyl]piperazin-1-yl}-N-acetyl-N-hydroxy-8-oxo octanamide as an crème coloured solid (0.04 g, 30.77%). ¹H NMR (400 MHz, CDCl₃): δ1.24 (2H, m, —CH₂), 1.39 (3H, t, —CH₃), 1.61 (2H, m, —CH₂), 2.23 (3H, s, —CH₃), 2.25 (2H, t, —CH₂), 2.37 (2H, t, —CH₂), 2.63 (4H, m, —CH₂), 3.32 (2H, s, —CH₂), 3.58 (2H, t, —CH₂), 3.73 (2H, t, —CH₂), 4.08 (2H, q, —CH₂), 7.04 (1H, d, —CH), 7.28 (1H, s, —CH), 7.67 (1H, d, —CH), 9.44 (1H, brs, —OH), 10.28 (1H, brs, —NH); M⁺+1 found at 534.0; m.p: 98-103° C.

The Following Compounds were Synthesized using the Procedure Given in the Example 37.

| | | |
|---|---|---|
| Ex. | Structure | Analytical Data |

| | | |
|---|---|---|
| 38 | 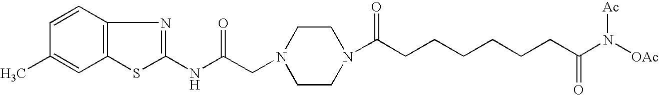 | ¹H NMR (400 MHz, CDCl₃): δ1.13 (2H, m, —CH₂), 1.27 (2H, m, —CH₂), 1.35 (4H, m, —CH₂), 1.65 (4H, m, —CH₂), 2.31 (3H, s, —CH₃), 2.36 (3H, s, —CH₃), 2.48 (3H, s, —CH₃), 2.63 (4H, t, —CH₂), 3.31 (2H, s, —CH₂), 3.58 (2H, t, —CH₂), 3.73 (2H, t, —CH₂), 7.27 (1H, d, —CH), 7.62 (1H, s, —CH), 7.67 (1H, d, |
| # —CH), 10.30 (1H, brs, —NH); M⁺ + 1 found at 546.2. | | |
| | Pale yellow solid, Hygroscopic | |
| 39 | 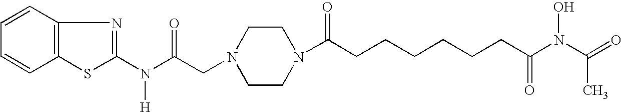 | ¹H NMR (400 MHz, CDCl₃): δ1.40 (4H, m, —CH₂), 1.68 (4H, m, —CH₂), 2.23 (3H, s, —CH₃), 2.25 (2H, t, —CH₂), 2.36 (2H, t, —CH₂), 2.64 (4H, m, —CH₂), 3.33 (2H, s, —CH₂), 3.59 (2H, t, —CH₂), 3.74 (2H, t, —CH₂), 7.44 (2H, dd, —CH), 7.82 (2H, dd, —CH), 9.45 (1H, brs, —NH); M⁺ + 1 found at 490.1. |
| 40 | 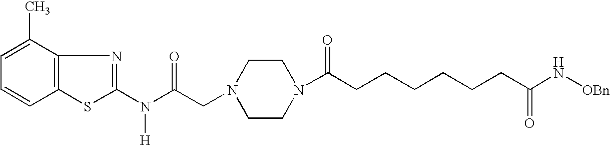 | ¹H NMR (400 MHz, CDCl₃): δ1.34 (4H, m, —CH₂), 1.66 (4H, m, —CH₂), 2.06 (2H, m, —CH₂), 2.32 (2H, t, —CH₂), 2.62 (4H, m, —CH₂), 2.66 (3H, s, —CH₃), 3.01 (2H, s, —CH₂), 3.62 (2H, t, —CH₂), 3.74 (2H, t, —CH₂), 4.92 (2H, s, —CH₂), 7.23 (3H, m, —CH), 7.38 (4H, m, —CH), 7.66 (1H, d, —CH), 8.39 (1H, brs, |
| # —NH), 10.33 (1H, brs, —NH); M⁺ + 1 found at 552.2. | | |
| | Pale brown sticky compound | |
| 41 | 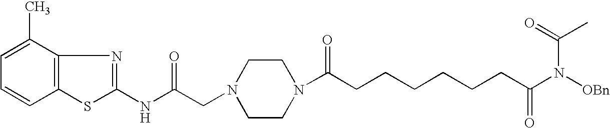 | ¹H NMR (400 MHz, CDCl₃): δ1.35 (4H, m, —CH₂), 1.58 (4H, m, —CH₂), 2.18 (3H, s, —CH₃), 2.30 (4H, m, —CH₂), 2.34 (4H, t, —CH₂), 2.66 (3H, s, —CH₃), 3.00 (2H, s, —CH₂), 3.60 (2H, t, —CH₂), 3.76 (2H, t, —CH₂), 5.02 (2H, s, —CH₂), 7.24 (3H, m, —CH), 7.33 (4H, m, —CH), 7.66 (1H, d, —CH), 10.29 (1H, brs, |
| # —NH); M⁺ + 1 found at 594.2. | | |
| | Pale brown sticky compound | |

### EXAMPLE 42

### Synthesis of 6-{4-[2-(1,3-Benzothiozol-2-ylamino)-2-oxoethyl]piperazin-1-yl}-N-{[2-amino-4-(methylthio)butanoyl]amino}hexanamide

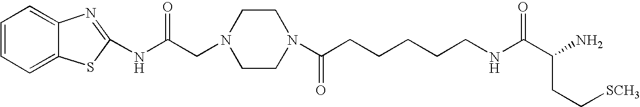 Step-I

### Synthesis of 6-[(t-Butoxycarbonyl)amino]hexanoic acid

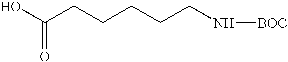

Aminocaproic acid (3 g, 22.87 mmol) taken in to water (25 ml), added sodium bicarbonate, stirred for 10 minutes and cooled to 0-5° C. In another flask BOC-anhydride was taken into 1,4-dioxane (25 ml), cooled to 0-5° C., added to the above reaction mixture and stirred the reaction mixture overnight. Extracted with ethylacetate (150 ml×1). The aqueous layer acidified with 1:1 aqueous. Hydrochloric acid up to pH 2 and extracted with ethylaceate (250 ml×2). The combined organic layer was washed with brine, dried over anhydrous Na₂SO₄ and concentrated under reduced pressure to afford a concentrate (5.1 g, 96.55%), which was used further for the next reaction.

Step-II

### Synthesis of 6-(4-Benzylpiperazin-1-yl)-N-[(tert-butoxycarbonyl)amino]hexanamide

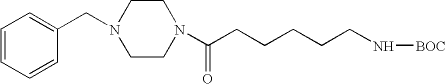

6-[(t-Butoxycarbonyl)amino]hexanoic acid (5.1 g, 22.10 mmol), 2-Amino-1,3-benzothiazole (3.32 g, 22.10 mmol), 1-(3-Dimethylaminopropyl)-3-ethylcarbodi-imide hydrochloride (EDCI) (7.63 g, 39.8 mmol), 1-Hydroxybenzotriazole (HOBt) (1.2 g, 8.84 mmol) were taken to tetrahydrofuran (50 ml) and stirred for 15 minutes N,N-diisopropylethyleneamine (DIPEA) (5.7 ml, 33.16 mmol) was added drop wise to the above and stirring was continued for 6 hours. Subsequently the THF was stripped off and water was added to the reaction mixture. It was extracted thrice with dichloromethane; the combined organic layer was washed with brine, dried over anhydrous Na₂SO₄ and concentrated under reduced pressure to give 6-(4-benzylpiperazin-1-yl)-N-[(tert-butoxycarbonyl)amino]hexanamide (6 g, 75%).

Step-III

### Synthesis of 6-(Piperazin-1-yl)-N-[(t-butoxycarbonyl)amino]hexanamide

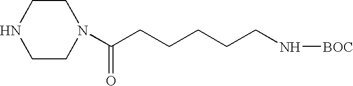

6-(4-Benzylpiperazin-1-yl)-N-[(t-butoxycarbonyl)amino]hexanamide (11 g) was dissolved in methanol (300 ml), added 20% of palladium on carbon (2.2 g, 10%) and stirred under hydrogen atmosphere for 6 hours. Filter the reaction mixture through celite bed and concentrated the filtrate under reduced pressure to get pure 6-(Piperazin-1-yl)-N-[(t-butoxycarbonyl)amino]hexanamide (6.8 g, 83.23%).

Step-IV

### 6-{4-[2-(1,3-Benzothiozol-2-ylamino)-2-oxoethyl]piperazin-1-yl}-N-[(tert-butoxycarbonyl)amino]hexanamide

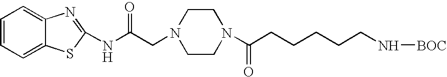

To a solution of N-1,3-benzothiazol-2-yl-2-bromoacetamide (1.75 g, 6.44 mmol) in dimethylsulfoxide (DMSO) (120 ml), added 6-(Piperazin-1-yl)-N-[(t-butoxycarbonyl)amino]hexanamide, and the reaction mixture was stirred for 15 hours and added cold water. It was extracted twice with ethyl acetate (300 ml); the combined organic layer was washed with brine, dried over anhydrous Na₂SO₄ and concentrated under reduced pressure to give crude 6-{4-[2-(1,3-Benzothiozol-2-ylamino)-2-oxoethyl]piperazin-1-yl}-N-[(tert-butoxycarbonyl)amino]hexanamide (2.7 g, 32.93%).

Step-V

### 6-{4-[2-(1,3-Benzothiozol-2-ylamino)-2-oxoethyl]piperazin-1-yl}hexanamine

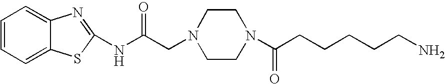

6-{4-[2-(1,3-Benzothiozol-2-ylamino)-2-oxoethyl]piperazin-1-yl}-N-[(t-butoxycarbonyl)amino]hexanamide (0.6 g, 1.23 mmol) was dissolved in dichloromethane (15 ml), added trifluoroacetic acid (TFA), (0.93 ml, 12.30 mmol) and stirred at room temperature for 3 hours subsequently the solvent was stripped off and washed with diisopropylether (50 ml×2) to get pure 6-{4-[2-(1,3-Benzothiozol-2-ylamino)-2-oxoethyl]piperazin-1-yl}hexanamine (0.42 g, 88%).

Step-VI

### Synthesis of 6-{4-[2-(1,3-Benzothiozol-2-ylamino)-2-oxoethyl]piperazin-1-yl}-N-{[2-(t-butoxycarbonyl)amino-3-hydroxypropanoyl]amino}hexanamide

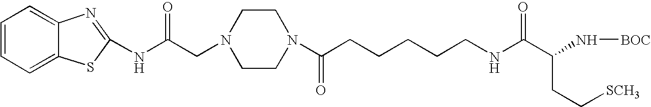

2-[(t-Butoxycarbonyl)amino]-4-(methylthio)butanoic acid (0.48 g, 1.93 mmol), 6-{4-[2-(1,3-Benzothiozol-2-ylamino)-2-oxoethyl]piperazin-1-yl}hexanamine (0.50 g, 1.29 mmol), 1-(3-Dimethylaminopropyl)-3-ethylcarbodi-imide hydrochloride (EDCI) (0.44 g, 2.31 mmol), 1-Hydroxybenzotriazole (HOBt) (0.07 g, 0.51 mmol) were taken to tetrahydrofuran (20 ml) and stirred for 15 minutes Triethyamine (Et₃N) (0.36 ml, 2.57 mmol) was added drop wise to the above and stirring was continued for 5 hours. The reaction mixture was diluted with ethylacetate (100 ml) and filtered through filter paper. The filtrate was washed with water (100 ml×2) and brine (100 ml), dried over anhydrous Na₂SO₄ and concentrated under reduced pressure to give crude extract of 6-{4-[2-(1,3-benzothiozol-2-ylamino)-2-oxoethyl]piperazin-1-yl}-N-{[2-(tert-butoxycarbonyl)amino-3-hydroxypropanoyl]amino}hexanamide (0.78 g, 116.42%)

Step-VII

### Synthesis of 6-{4-[2-(1,3-Benzothiozol-2-ylamino)-2-oxoethyl]piperazin-1-yl}-N-{[2-amino-4-(methylthio)butanoyl]amino}hexanamide

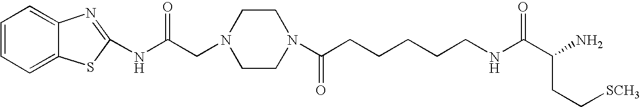

6-{4-[2-(1,3-Benzothiozol-2-ylamino)-2-oxoethyl]piperazin-1-yl}-N-{[2-(t-butoxycarbonyl)amino-3-hydroxypropanoyl]amino}hexanamide(0.75 g,1.44 mmol) was dissolved in dichloromethane (5 ml), added trifluoroacetic acid (TFA), (1.66 ml, 21.68 mmol) and stirred at room temperature for 2 hours subsequently the solvent was stripped off and washed with diisopropylether (50 ml×2) The resulting crude material was purified by column chromatography using methanol and dichloromethane mixture to give pure 6-{4-[2-(1,3-Benzothiozol-2-ylamino)-2-oxoethyl]piperazin-1-yl}-N-{[2-amino-4-(methylthio)butanoyl]amino}hexanamide as crème coloured solid (0.48 g, 79.34%o). ¹H NMR (400 MHz, DMSO): δ1.25 (2H, m, —CH₂), 1.48 (4H, m, —CH₂), 1.8 (2H, m, —CH₂), 2.01 (2H, t, —CH₂), 2.05 (3H, s, —CH₃), 2.30 (2H, t, —CH₂), 2.44 (4H, m, —CH₂), 3.35 (2H, t, —CH₂), 3.40 (4H, m, —CH₂), 3.49 (2H, s, —CH₂), 3.60 (1H, t, —CH), 7.32 (1H, m, —CH), 7.45 (1H, m, —CH), 7.76 (1H, d, —CH), 8.0 (1H, d, —CH), 8.13 (2H, brs, —NH₂), 8.42 (11H, brs, —NH), 12.15 (1H, brs, —NH); M⁺+1 found at 521.2; m.p.: 82-85° C.

The Following Compounds were Synthesized using the Procedure given in the Example 42.

| Ex. | Structure | Analytical Data |
|---|---|---|
| 43 | 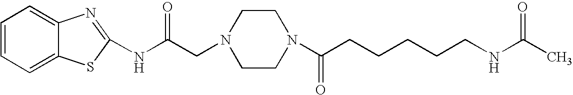 | ¹H NMR (400 MHz, DMSO): δ 1.53 (2H, m, —CH₂), 1.65 (4H, —CH₂), 1.98 (3H, s, —CH₃), 2.35 (2H, m, —CH₂), 2.65 (4H, t, —CH₂), 3.26 (2H, m, —CH₂), 3.34 (2H, s, —CH₂), 3.60 (2H, t, —CH₂), 3.74 (2H, t, —CH₂), 5.90 (1H, brs, —NH), 7.34 (1H, d, —CH), 7.46 (1H, d, —CH), 7.82 (2H, m, —CH), 11.85 (1H, brs, —NH); M⁺ + 1 |
| # found at 432.1. | | |
| | Oily compound | |
| 44 | 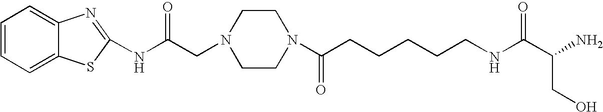 | ¹H NMR (400 MHz, DMSO): δ 1.29 (2H, t, —CH₂), 1.47 (4H, m, —CH₂), 2.29 (2H, t, —CH₂), 3.11 (2H, m, —CH₂), 3.35 (2H, t, —CH₂), 3.48 (4H, m, —CH₂), 3.72 (1H, m, —CH), 4.10 (2H, t, —CH₂), 5.46 (1H, brs, —OH), 7.31 (1H, m, —CH), 7.45 (1H, m, —CH), 7.75 (1H, d, —CH), 7.99 (1H, d, —CH), 8.02 (2H, brs, —NH₂), 8.32 (1H, |
| # brs, —NH), 12.17 (1H, brs, —NH); M⁺ + 1 found at 477.1; m.p.: 82-85° C. | | |
| | Oily compound | |
| 45 | 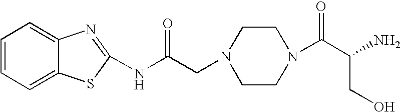 | ¹H NMR (400 MHz, DMSO): δ 2.55 (4H, m, —CH₂), 3.37 (2H, s, —CH₂), 3.58 (2H, d, —CH₂), 3.70 (4H, m, —CH₂), 4.39 (1H, t, —CH), 5.47 (1H, brs, —OH), 7.32 (1H, m, —CH), 7.45 (1H, m, —CH), 7.76 (1H, d, —CH), 8.0 (1H, d, —CH), 8.09 (2H, brs, —NH), 12.26 (1H, brs, —NH); M⁺ + 1 found at 364.1. |
| | Brown solid, hygroscopic | |
| 46 | 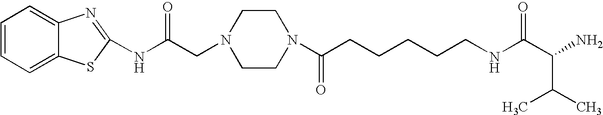 | ¹H NMR (400 MHz, DMSO): δ 0.88 (6H, m, —CH₃), 1.29 (2H, t, —CH₂), 1.47 (4H, m, —CH₂), 1.99 (1H, m, —CH), 2.30 (2H, t, —CH₂), 2.5 (4H, m, —CH₂), 3.20 (2H, m, —CH₂), 3.23 (1H, d, —CH), 3.33 (2H, s, —CH₂), 3.48 (4H, m, —CH₂), 7.31 (1H, m, —CH), 7.44 (1H, m, —CH), 7.60 (1H, brs, —NH), 7.75 (1H, d, —CH), 7.99 (1H, |
| # d, —CH), 8.11 (2H, brs, —NH₂); M⁺ + 1 found at 489.2. | | |
| | Hygroscopic | |
| 47 | 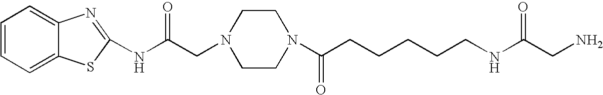 | ¹H NMR (400 MHz, DMSO): δ 1.29 (2H, t, —CH₂), 1.47 (4H, m, —CH₂), 2.30 (2H, t, —CH₂), 2.5 (4H, m, —CH₂), 3.12 (2H, m, —CH₂), 3.36 (2H, s, —CH₂), 3.51 (2H, s, —CH₂), 3.60 (4H, m, —CH₂), 7.32 (1H, m, —CH), 7.45 (1H, m, —CH), 7.75 (1H, d, —CH), 7.97 (1H, d, —CH), 8.0 (2H, brs, —NH₂), 12.2 (1H, brs, —NH); M⁺ + 1 |
| # found at 447.1. | | |
| | Pale yellow sticky compound | |
| 48 | 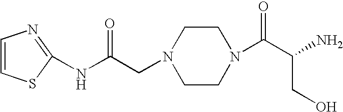 | ¹H NMR (400 MHz, DMSO): δ 2.50 (4H, m, —CH₂), 3.35 (2H, s, —CH₂), 3.56 (2H, d, —CH₂), 3.65 (4H, m, —CH₂), 4.5 (1H, t, —CH), 5.42 (1H, brs, —OH), 7.26 (1H, d, —CH), 7.48 (1H, d, —CH), 7.89 (1H, d, —CH), 8.1 (2H, brs, —NH); M⁺ + 1 found at 314.1. |

### Anti-cancer experimental methods

Anti-Cancer Screen:

Experimental drugs are screened for anti-cancer activity in three cell lines for their GI₅₀, TGI and LC₅₀ values (using five concentrations for each compound). The cell lines are maintained in DMEM containing 10% fetal bovine serum. 96 well micro titer plates are inoculated with cells in 100 μL for 24 h at 37° C., 5% CO2, 95% air and 100% relative humidity. 5000 HCT 116 cells/well, 5000 NCIH 460 cells/well and 5000 U251 cells/well are plated. A separate plate with these cell lines is also inoculated to determine cell viability before the addition of the compounds (T₀).

Addition of Experimental Drugs:

Following 24-hour incubation, experimental drugs are added to the 96 well plates. Each plate contains one of the above cell lines and the following in triplicate: five different concentrations (0.01, 0.1, 1, 10 and 100 μM) of four different compounds, appropriate dilutions of a cytotoxic standard and control (untreated) wells. Compounds are dissolved in DMSO to make 20 mM stock solutions on the day of drug addition and frozen at −20° C. Serial dilutions of these 20 mM stock solutions are made in complete growth medium such that 100 μL of these drug solutions in medium, of final concentrations equaling 0.01, 0.1, 1, 10 and 100 μM can be added to the cells in triplicate. Standard drugs whose anti-cancer activity has been well documented and which are regularly used are doxorubicin and SAHA.

End-Point Measurement:

For To measurement, 24 hours after seeding the cells, 10 μL of 3-(4,5-Dimethyl-2-thiazolyl)-2,5-diphenyl-2H-tetrazolium (MTT) solution per well is added and incubation carried out for 3 hours at 37° C., 5% CO₂, 95% air and 100% relative humidity, protected from light. Cells incubated with compounds for 48 hours are treated similarly except with the addition of 20 μL MTT solution per well and a subsequent incubation under the same conditions. After 3 hours of MTT incubation, well contents are aspirated carefully followed by addition of 150 μL DMSO per well. Plates are agitated to ensure solution of the formazan crystals in DMSO and absorbance read at 570 nm.

Calculation of GI₅₀, TGI and LC₅₀:

Percent growth is calculated for each compound's concentration relative to the control and zero measurement wells (T₀; viability right before compound addition).
If a test well's O.D. value is greater than the To measurement for that cell line % Growth=(test−zero)/(control−zero)×100
If a test well's O.D. value is lower than the To measurement for that cell line, then, % Growth=(test−zero)/zero×100
Plotting % growth versus experimental drug concentration, GI₅₀ is the concentration required to decrease % growth by 50%; TGI is the concentration required to decrease % growth by 100% and LC₅₀ is the concentration required to decrease % growth by 150%.
   HDAC Activitv Screening:
   Histone Deacetylase (HDAC) Inhibition Assay using Boc-Lys (Ac)-AMC Substrate:

Inhibition of HDAC has been implicated to modulate transcription and to induce apoptosis or differentiation in cancer cells. The fluorometric assay provides a fast and fluorescence based method that eliminates radioactivity, extractions or chromatography, as used in traditional assays. The assay based on two steps. First, the HDAC fluorometric substrate, which comprises an acetylated lysine side chain, is incubated with a sample containing HDAC activity (Mouse Liver Extract). Deacetylation of the substrate sensitizes the substrate, in the second step; treatment with the Trypsin stop solution produces a fluorophore that can be easily analyzed using fluorescence plate reader.

Assay was done in 96 well black microplate and total volume of the assay is 100 ul. Mouse liver enzyme is diluted 1:6 with HDAC buffer. Enzyme cocktail made of 10 ul of diluted enzyme and 30 ul of HDAC buffer. 40 ul of enzyme cocktail dispensed into each well. 10 ul of different concentrations of inhibitor added in to each well, except enzyme control well. Preincubated the plate at 30° C. for 5 minutes. The HDAC reaction is started by adding 50 ul of HDAC substrate (Boc-Lys (Ac)-AMC Substrate) solution. Incubated the plate at 30° C. for 30 minutes. Adding 100 ul of Trypsin stop solution stops the reaction. The plate is incubated again at 30° C. for 20-30 minutes. The release of AMC is monitored by measuring the fluorescence at excitation wavelength of 365 or 360 nm and emission wavelength of 440 or 460 nm. Buffer and substrate alone kept for blank subtraction. (Dennis Wegener et al, Anal. Biochem, 321, 2003, 202-208). Results are indicated in the following Table.

TABLE

| S. | NCIH460 | | | HCT116 | | | MDAMB231/U251 | | | Mean | HDAC Inhibition | | |
|---|---|---|---|---|---|---|---|---|---|---|---|---|---|
| No. | GI₅₀ | TGI | LC₅₀ | GI₅₀ | TGI | LC₅₀ | GI₅₀ | TGI | LC₅₀ | GI₅₀ | 1 μM | 10 μM | IC₅₀ |
| 1. | >100 | >100 | >100 | >100 | >100 | >100 | 80 | >100 | >100 | >100 | 10.48 | 38.59 | NA |
| 2. | >100 | >100 | >100 | >100 | >100 | >100 | >100 | >100 | >100 | >100 | 8.31 | 34.56 | NA |
| 3. | >100 | >100 | >100 | 49 | 99 | >100 | 48 | >100 | >100 | >100 | 42.40 | 86.74 | NA |
| 4. | 9 | 91 | >100 | 60 | >100 | >100 | 45 | >100 | >100 | 38 | 15.82 | 50.88 | NA |
| 5. | >100 | >100 | >100 | >100 | >100 | >100 | >100 | >100 | >100 | >100 | 53.50 | 81.00 | 0.065 |
| 6. | 0.8 | 53 | >100 | 4.5 | 42.0 | >100 | 0.01 | 0.4 | >100 | 2.00 | 63.40 | 83.30 | 0.062 |
| 7. | 50 | 88 | >100 | 85 | >100 | >100 | >100 | >100 | >100 | 67.50 | 27.76 | 45.96 | NA |
| 8. | 18 | >100 | >100 | 7 | 80 | >100 | >100 | >100 | >100 | 12.50 | 31.41 | 69.85 | ND |
| 9. | >100 | >100 | >100 | >100 | >100 | >100 | >100 | >100 | >100 | >100 | 3.39 | 23.65 | NA |
| 10. | 0.5 | 2.0 | 95 | 3.0 | 50 | >100 | 2.0 | 9.0 | >100 | 1.80 | 54.06 | 87.16 | 1.5 |
| 11. | 0.3 | 0.9 | 8.0 | 5.0 | 40 | >100 | 1.0 | 8.0 | >100 | 2.10 | 31.75 | 65.63 | 6.5 |
| 12. | 25 | 75 | >100 | 7.0 | >100 | 100 | 19 | 65 | >100 | 17 | 57.31 | 85.59 | ND |
| 13. | >100 | >100 | >100 | 6.0 | >100 | >100 | 6.0 | 30 | >100 | >100 | 26.73 | 55.56 | NA |
| 14. | >100 | >100 | >100 | 50 | >100 | >100 | 0.08 | 27 | >100 | >100 | 23.11 | 57.33 | NA |
| 15. | >100 | >100 | >100 | 7.0 | >100 | >100 | 0.05 | 10 | >100 | >100 | 24.02 | 58.39 | NA |
| 16. | 30 | 52 | >100 | 58 | >100 | >100 | 1.0 | >100 | >100 | 29.67 | 29.26 | 68.52 | NA |
| 17. | 7.5 | >100 | >100 | 26 | >100 | >100 | 67 | >100 | >100 | 33.50 | 68.04 | 96.19 | ND |
| 18. | 30 | >100 | >100 | 63 | >100 | >100 | >100 | >100 | >100 | 46.50 | 65.15 | 94.50 | ND |
| 19. | >100 | >100 | >100 | >100 | >100 | >100 | 57 | >100 | >100 | >100 | 18.82 | 51.25 | NA |
| 20. | >100 | >100 | >100 | >100 | >100 | >100 | >100 | >100 | >100 | >100 | 26.4 | 55.4 | NA |
| 21. | 93 | >100 | >100 | 52 | >100 | >100 | 38 | >100 | >100 | 61 | 82.9 | 96.2 | 0.57 |
| 22. | >100 | >100 | >100 | 67 | >100 | >100 | >100 | >100 | >100 | >100 | 6.0 | 50.0 | NA |
| 23. | >100 | >100 | >100 | 63 | >100 | >100 | 38 | 78 | >100 | >100 | 47.2 | 81.7 | 4.8 |
| 24. | 60 | >100 | >100 | 0.70 | >100 | >100 | 5 | 66 | >100 | 21.90 | 4 | 27.4 | NA |
| 25. | 9 | >100 | >100 | 3.5 | >100 | >100 | 0.07 | 0.80 | >100 | 4.2 | NA | 3.6 | NA |
| 26. | 9.50 | 92 | >100 | 8.60 | >100 | >100 | 27 | 85 | >100 | 15 | NA | 4.5 | NA |
| 27. | 8 | >100 | >100 | 8 | >100 | >100 | 28 | 80 | >100 | 14.70 | NA | NA | NA |
| 28. | 21 | >100 | >100 | 5.5 | 58 | >100 | 0.08 | 9 | >100 | 8.86 | NA | NA | NA |
| 29. | 0.8 | 93 | >100 | 30 | >100 | >100 | 11 | 55 | 95 | 13.9 | 61.41 | 97.00 | 0.76 |
| 30. | 42.5 | 80.5 | >100 | 8 | 51 | 79 | 35 | 56 | 82 | 28.50 | NA | 4.65 | NA |
| 31. | 60 | >100 | >100 | 60 | >100 | >100 | 0.60 | 94 | >100 | 40.20 | 45.20 | 84.96 | ND |
| 32. | 38 | 72 | >100 | 7.0 | 42 | 95 | 1.50 | 9.5 | 98 | 15.50 | 60.30 | 98.91 | ND |
| 33. | 90 | >100 | >100 | 55 | >100 | >100 | 10 | 98 | >100 | 51.67 | 50.09 | 92.38 | ND |
| 34. | >100 | >100 | >100 | 50 | >100 | >100 | 6.0 | 74 | >100 | 28 | 45.65 | 89.86 | ND |
| 35. | 56 | 100 | >100 | 8.0 | 40 | 92 | 5.0 | 45 | >100 | 23 | 68.3 | 96.93 | ND |
| 36. | 52 | >100 | >100 | 40 | 82 | >100 | 27 | 62 | >100 | 39.67 | 98.2 | 72.99 | 0.95 |
| 37. | 40 | 74 | >100 | 7.0 | 44 | >100 | 4.0 | 45 | 95 | 17.00 | 55.57 | 94.70 | ND |
| 38. | 40 | >100 | >100 | 25 | 88 | >100 | 0.4 | 30 | >100 | >100 | 37.83 | 84.23 | ND |
| 39. | 44 | 48 | >100 | 8 | 57 | 98 | 22 | 52 | 78 | 24.7 | 81.4 | 96.6 | 0.55 |
| 40. | 24 | 85 | >100 | 4.5 | 32 | >100 | 0.10 | 0.50 | >100 | 9.5 | NA | 4.1 | NA |
| 41. | 26 | 100 | >100 | 4.5 | 53 | >100 | 0.1 | 6.2 | >100 | 10.2 | 0.6 | NA | NA |
| 42. | 56 | >100 | >100 | 26 | 78 | >100 | 99.5 | >100 | >100 | 61 | ND | ND | ND |
| 43. | >100 | >100 | >100 | 90 | >100 | >100 | >100 | >100 | >100 | >100 | 2.61 | 5.24 | NA- |
| 44. | >100 | >100 | >100 | >100 | >100 | >100 | >100 | >100 | >100 | >100 | 5.45 | 1.79 | NA |

ND = Not Done;
NA = No Activity

1. A compound of formula (I), 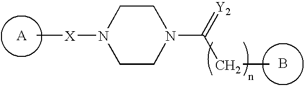
derivatives, analogs, tautomeric forms, stereoisomers, polymorphs, pharmaceutically acceptable salts and compositions thereof; wherein, suitable groups represented by A represents substituted or unsubstituted groups selected from aryl group comprising phenyl, naphthyl and the like; arylalkyl comprising benzyl, phenylethyl, phenylpropyl and the like; heterocyclyl groups comprising pyrrolidinyl, thiazolidinyl, oxazolidinyl, morpholinyl, thiomorpholinyl, piperidinyl, piperazinyl; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like; benzofused heteroaryl groups comprising indolyl, indolinyl, benzothiazolyl, quinoline, quinoxaline, quinazoline, acridine, phenazine pteridinyl, phenoxazine, phenothiazine, carbazolyl and the like; the group X is represented by 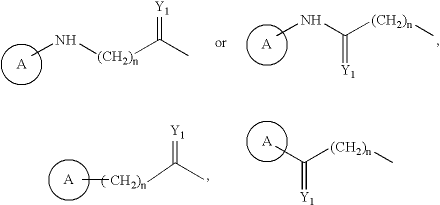
or X and A are fused to form a cyclic structure; Y₁ and Y₂ may be same or different and independently represent oxygen or sulphur; wherein B represents one or more substituted linear or branched alkyl groups substituted by groups comprising amino, hydroxyl, thiol and the like; thioate, thiol, substituted or unsubstituted hydroxyal amine group (O-acetyl, N-acetyl, O-benzyl both or either of any two combination), substituted or unsubstituted hydroxamic acid group (O-acetyl, N-acetyl, O-benzyl both or either of any two combination), carbonyl or amino group substituted with acetyl group or amino acids or their corresponding hydroxamic acids or their esters (viz. isomers of amandine, cystine, glycine, valine, methionine, serine, tyrosine, isonipecolic acid, proline, and the like.) and n is an integer in the range of 0 to 7; suitable groups substituted on A with one or more same or different substituents selected from a group consisting of halogens (fluorine, chlorine, bromine, iodine), hydroxy, nitro, cyano, azido, nitroso, amino, hydrazine, formyl, alkyl, haloalkyl, haloalkoxy, cycloalkyl, aryl (which is further substituted), alkoxy, aryloxy, acyl, ecology, acyloxyacyl, heterocyclyl, heteroaryl (is further substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives; furthermore A which is a cyclic ring represents substituted or unsubstituted 5 to 10 membered ring systems, and also the rings are monocyclic or bicyclic, saturated or partially saturated or aromatic containing 1 to 4 hetero atoms selected from O, S and N and the like.

2. The compound of claim 1, selected from a group consisting of:
N-{2-[4-(5-Thioacetylpentanoyl)piperazin-1-yl]-2-oxoethyl}4-methyl-1,3-benzothiazol-2-amine;
N-{2-[4-(6-Thioacetylhexanoyl)piperazin-1-yl]-2-oxoethyl}-morpholine;
N-{2-[4-(6-Thioacetylhexanoyl)piperazin-1-yl]-2-oxoethyl}-6-methyl-1,3-benzothiazol-2-amine;
2-[4-(5-Thioacetylpentanoyl)piperazin-1-yl]-N-(4-methyl-1,3-thiazol-2-yl)acetamide;
2-[4-(6-Thioacetylhexanoyl)piperazin-1-yl]-N-(4-nitro-1,3-thiazol-2-yl)acetamide;
2-[4-(6-Thioacetylhexanoyl)piperazin-1-yl]-N-(1,3-benzothiazol-2-yl)acetamide;
2-[4-(5-Thioacetylpentanoyl)piperazin-1-yl]-N-(1,3-thiozole-2-yl)acetamide;
2-[4-(5-Thioacetylpentanoyl)piperazin-1-yl]-N-(1,3-benzothiazol-2-yl)acetamide;
2-[4-(5-Thioacetylpentanoyl)piperazin-1-yl]-N-(4-nitro-1,3-thiazol-2-yl)acetamide;
2-[4-(6-Thioacetylhexanoyl)piperazin-1-yl]-N-(4-methyl-1,3-benzothiazol-2-yl)acetamide;
2-[4-(5-Thioacetylpentanoyl)piperazin-1-yl]-N-(6-methyl-1,3-benzothiazol-2-yl)acetamide;
2-[4-(6-Thioacetylhexanoyl)piperazin-1-yl]-N-(6-methyl-1,3-benzothiazol-2-yl)acetamide;
2-[4-(5-Thioacetylpentanoyl)piperazin-1-yl]-N-(4-methyl-1,3-benzothiazol-2-yl)acetamide;
2-[4-(5-Thioacetylpentanoyl)piperazin-1-yl]-N-(4-tertiarybutyl-1,3-thiazol-2-yl)acetamide;
2-[4-(5-Thioacetylpentanoyl)piperazin-1-yl]-N-(6-ethoxy-1,3-benzothiazol-2-yl)acetamide;
2-[4-(5-Thioacetylpentanoyl)piperazin-1-yl]-N-(phenyl-2-yl)acetamide;
2-[4-(6-Thioacetylhexanoyl)piperazin-1-yl]-N-(4-methyl-1,3-thiazol-2-yl)acetamide;
2-[4-(6-Thioacetylhexanoyl)piperazin-1-yl]-N-(phenyl-2-yl)acetamide;
2-[4-(6-Thioacetylhexanoyl)piperazin-1-yl]-N-(morpholine)acetamide;
2-[4-(5-Thioacetylpentanoyl)piperazin-1-yl]-N-(3,5-dimethoxybenzyl)-2-yl)acetamide;
2-[4-(6-Thioacetylhexanoyl)piperazin-1-yl]-N-(4-methoxy-1,3-benzothiazol-2-yl)acetamide;
2-[4-(6-Thioacetylhexanoyl)piperazin-1-yl]-N-(6-fluorophenyl-2-yl)acetamide;
2-[4-(5-Thioacetylpentanoyl)piperazin-1-yl]-N-(6-fluorophenyl-2-yl)acetamide;
N-1,3-benzothiazol-2-yl-2-[4-(6-mercaptohexanoyl)piperazin-1-yl]acetamide;
N-6-methyl-1,3-benzothiazol-2-yl-2-[4-(6-mercaptohexanoyl)piperazin-1-yl]acetamide;
N-1,3-benzothiazol-2-yl-2-{6-[6 (hydroxyamino)hexanoyl]piperazin-1-yl}acetamide;
N-4-methyl-1,3-benzothiazol-2-yl-2-{6-[6 (hydroxyamino)hexanoyl]piperazin-1-yl}acetamide;
N-4-methoxy-1,3-benzothiazol-2-yl-2-{6-[6 (N-acetyl-O-benzyl)hexanoyl]piperazin-1-yl}acetamide;
8-{4-[2-(1,3-Benzothiazol-2-ylamino)-2-oxoethyl]piperazin-1-yl}-N-hydroxy-8-oxo octanamide;
8-{4-[2-(6-Methyl-1,3-benzothiazol-2-ylamino)-2-oxoethyl]piperazin-1-yl}-N—N-[(2-amino-3-hydroxypropanoyl)amino]-8-oxo octanamide;
8-{4-[2-(Phenyl-2-ylamino)-2-oxoethyl]piperazin-1-yl}-N-hydroxy-8-oxo octanamide;
8-{4-[2-(6-Ethoxy-1,3-benzothiazol-2-ylamino)-2-oxoethyl]piperazin-1-yl}-N-hydroxy-8-oxo octanamide;
8-{4-[2-(4-Methyl-1,3-thiazol-2-ylamino)-2-oxoethyl]piperazin-1-yl}-N-hydroxy-8-oxo octanamide;
8-{4-[2-(1,3-Thiazol-2-ylamino)-2-oxoethyl]piperazin-1-yl}-N-hydroxy-8-oxo octanamide;
8-{4-[2-(6-Methyl-1,3-benzothiazol-2-ylamino)-2-oxoethyl]piperazin-1-yl}-N-hydroxy-8-oxo octanamide;
8-{4-[2-(3-Fluorophenyl-2-ylamino)-2-oxoethyl]piperazin-1-yl}-N-hydroxy-8-oxo octanamide;
8-{4-[2-(6-Ethoxy-1,3-benzothiazol-2-ylamino)-2-oxoethyl]piperazin-1-yl}-N-acetyl-N-hydroxy-8-oxo octanamide;
8-{4-[2-(6-Methyl-1,3-benzothiazol-2-ylamino)-2-oxoethyl]piperazin-1-yl}-N-acetyl-N-hydroxyacetyl-8-oxo octanamide;
8-{4-[2-(1,3-Benzothiazol-2-ylamino)-2-oxoethyl]piperazin-1-yl}-N-acetyl-N-hydroxy-8-oxo octanamide;
8-{4-[2-(4-Methyl-1,3-benzothiazol-2-ylamino)-2-oxoethyl]piperazin-1-yl}-N-hydroxybenzyl-8-oxo octanamide;
8-{4-[[2-(4-Methyl-1,3-benzothiazol-2-ylamino)-2-oxoethyl]piperazin-1-yl}-N-acetyl-N-hydroxybenzyl-8-oxo octanamide;8-[4-(thiazole-2-aminoacetyl)piperazin-1-yl]-N-hydroxy-8-oxooctanamide;
Synthesis of 6-{4-[2-(1,3-Benzothiozol-2-ylamino)-2-oxoethyl]piperazin-1-yl}-N-{[2-amino-4-(methylthio)butanoyl]amino}hexanamide;
Synthesis of 6-{4-[2-(1,3-Benzothiozol-2-ylamino)-2-oxoethyl]piperazin-1-yl}-N-(acetyl amino)hexanamide;
6-{4-[2-(1,3-Benzothiozol-2-ylamino)-2-oxoethyl]piperazin-1-yl}-N-[(2-amino-3-hydroxypropanoyl)amino]hexanamide;
2-[4-(2-Amino-3-hydroxypropanoyl)piperazin-1-yl]-N-1,3-benzothiazol-2-ylacetamide;
6-{4-[2-(1,3-Benzothiozol-2-ylamino)-2-oxoethyl]piperazin-1-yl}-N-[(2-amino-3-methylbutyl)amino]hexanamide;
6-{4-[2-(1,3-Benzothiozol-2-ylamino)-2-oxoethyl]piperazin-1-yl}-N-(aminoacetyl)hexanamide and
2-[4-(2-Amino-3-hydroxypropanoyl)piperazin-1-yl]-N-1,3-thiazol-2-ylacetamide.

3. A pharmaceutical composition comprising a compound of formula (I) according to claimed in claim 1, as an active ingredient along with a pharmaceutically acceptable carrier, diluent, excipient or solvate.

4. The pharmaceutical composition according to claim 3, wherein the said composition is in the form of a tablet, capsule, powder, syrup, solution, aerosol or suspension.

5. The pharmaceutical composition as claimed in claim 3, wherein the amount of the compound of claim 1 in the composition is less than 70% by weight.

6. A method for modulating the number of biological processes, including transcription, cell cycle and regulating nucleosomal integrity.

7. A method of treating diseases that are involved in cellular growth such as malignant tumors, autoimmune diseases, skin diseases, infections etc

8. A method of inhibiting HDAC in a cell comprising treating said cell with an effective amount of a compound according to claim 1.

9. A method for the treatment of a condition mediated by HDAC comprising administering to a subject suffering from a condition mediated by HDAC a therapeutically effective amount of a compound according to the claim 1.

10. A method for the treatment of psoriasis comprising administering to a subject suffering from psoriasis a therapeutically effective amount of a compound according to claim 1.

11. A method for the treatment of a proliferative condition comprising administering to a subject suffering from a proliferative condition a therapeutically effective amount of a compound according to claim 1, to the mammal in need thereof.

12. A method for the treatment and/or prevention of cancer comprising administering to a subject suffering from cancer a therapeutically effective amount of a compound according to claim 1, to the mammal in need thereof.

13. A method for the treatment of psoriasis comprising administering to a subject suffering from psoriasis a therapeutically effective amount of a compound according to claim 2.

14. A method for the treatment of psoriasis comprising administering to a subject suffering from psoriasis a therapeutically effective amount of a compound according to claim 3.

15. A method for the treatment of psoriasis comprising administering to a subject suffering from psoriasis a therapeutically effective amount of a compound according to claim 4.

16. A method for the treatment of psoriasis comprising administering to a subject suffering from psoriasis a therapeutically effective amount of a compound according to claim 5.

17. A method for the treatment of psoriasis comprising administering to a subject suffering from psoriasis a therapeutically effective amount of a compound according to claim 6.
